SEQUENCE LISTING (7 sequences)

SEQ ID NO: 1 - DNA, 25 bp, Artificial Sequence
  antisense oligo cdh5-MO
tacaagaccgtctacctttccaatc

SEQ ID NO: 2 - DNA, 25 bp, Artificial Sequence
  antisense oligo sih-MO
catgtttgctctgatctgacacgca

SEQ ID NO: 3 - DNA, 25 bp, Artificial Sequence
  antisense oligo piezo1-MO
caaagttcagttcagctcacctcat

SEQ ID NO: 4 - DNA, 25 bp, Artificial Sequence
  antisense oligo dnmt3bb.1-MO1
ttatttcttccttcctcatcctgtc

SEQ ID NO: 5 - DNA, 25 bp, Artificial Sequence
  antisense oligo dnmt3bb.1-MO2
ctctcatctgaaagaatagcagagt

SEQ ID NO: 6 - DNA, 24 bp, Artificial Sequence
  PCR Primer 5′ of DH beta2.1
gtaggcacctgtggggaagaaact

SEQ ID NO: 7 - DNA, 25 bp, Artificial Sequence
  PCR Primer 3′ of JH beta2.7
tgagagctgtctcctactatcgatt

(19) United States
(12) Patent Application Publication
(10) Pub. No.: US 2023/0041065 A1
(43) Pub. Date: Feb. 9, 2023

(54) METHODS FOR GENERATING HEMATOPOIETIC STEM CELLS

(71) Applicant: The Brigham and Women`s Hospital, Inc., Boston, MA (US)

(72) Inventors: Dhvanit I. Shah, Malden, MA (US); Giorgia Scapin, Boston, MA (US)

(21) Appl. No.: 17/783,694

(22) PCT Filed: Dec. 9, 2020
(86) PCT No.: PCT/US20/63901

§ 371 Date: Jun. 9, 2022

Related U.S. Application Data
(60) Provisional application No. 62/945,838, filed on Dec. 9, 2019.

(51) Int. Cl. C12N 5/0789; A61K 35/28

(52) U.S. Cl. CPC C12N 5/0647; A61K 35/28; C12N 2506/28; C12N 2506/45; C12N 2506/11; C12N 2527/00

(57) ABSTRACT

In the various aspects and embodiments, this disclosure provides genetic, pharmacological, and mechanical stimuli for transitioning endothelial cells to hemogenic endothelial (HE) cells, and for transitioning HE cells to HSCs, including HSCs that comprise a significant level of LT-HSCs. The disclosure further provides methods for expanding HSCs using the genetic, pharmacological, and mechanical stimuli.

### CLAIM OF PRIORITY

This application claims the benefit of U.S. Provisional Patent Application Ser. No. 62/945,838, filed on Dec. 9, 2019. The entire contents of the foregoing are hereby incorporated by reference.

### FEDERALLY SPONSORED RESEARCH

This invention was made with Government support under Grant No. HL131645, awarded by the National Institute of Health. The Government has certain rights in the invention.

### BACKGROUND

Hematopoietic stem cells (HSCs) are derived during embryogenesis in distinct regions where specific inductive events convert mesoderm to blood stem cells and progenitors. HSCs can give rise to red cells, platelets, myeloid and lymphoid (T- & B-cells) cells in a process called hematopoiesis.

HSC transplantation (HSCT) is widely used to treat patients with blood, bone marrow, metabolic, and immune diseases. Despite advances in umbilical cord and haplo-identical stem cell transplantation, the therapeutic use of HSC transplantation is often restricted due to the difficulty of finding suitable human leukocyte antigen (HLA)-matched donors in a timely manner, especially in countries with ethnic minorities and lack of national unrelated donor registries. Although mixed-race people account for 1.6 percent (9.7 million) of the U.S. population, multiracial volunteers make up only 3 percent (21,000) of the 7 million people on the registry, leaving 6,000 patients without a bone marrow match. Even if one finds a suitable match, immunologic complications such as graft-versus-host disease (GVHD), donor rejection, and high treatment-related mortality could compromise patient survival. However, these complications are eliminated by autologous transplant. Although autologous HSCs would not replace allogeneic HSCs entirely, especially in the context of hematologic malignancy, they would overcome major hurdles in HSCT including, lack of donor availability and GVHD for patients with a broad span of malignant and non-malignant hematologic, immune, and metabolic disorders.

Thus, there is a need for generating HSCs, including autologous HSCs or off-the-shelf HSCs, for HSCT.

### SUMMARY

The present disclosure is based at least in part on the discovery that changes in expression or activity of certain endothelial and hematopoietic genes induces hematopoietic stem cell (HSC) formation from endothelial cells, including the formation of substantial numbers of Long Term (LT)-HSCs that can self-renew, engraft, and reconstitute multi-lineage adult blood.

cdh5-morphant (cdh5-MO) embryos have a heartbeat-mediated pulsation in blood vessels without cardiac output and active blood flow. Pulsation-derived stretching activates Piezo1 mechanosensitive channels that further enhance Dnmt3b expression in the aorta-gonad-mesonephros (AGM) region, and which in turn modulates expression of core endothelial and hematopoietic genes and their regulators to stimulate the hemogenic endothelial-to-HSC transition. The simulation of pulsation or the pharmacological activation of Piezo1 also yields two- to three times higher amounts of LT-HSCs, which reconstitute to normal, functional multi-lineage adult blood upon serial transplantation. In some embodiments, the hematopoietic stem cells produced according to this disclosure comprise substantial numbers of LT-HSCs, which exhibit superior engraftment, and reconstitute to functional, multi-lineage adult blood in the recipient.

In some embodiments, the invention provides a method of preparing a population of hematopoietic stem cells (HSCs) comprising LT-HSCs. The method comprises providing a population of cells comprising endothelial and/or hemogenic endothelial (HE) cells, and decreasing expression or modifying activity of two, three, four, five, or more endothelial genes selected from vegfa, hey2, grp116, gnal3, sox17, cdh5, plxnd1, bcl6, and apln in the endothelial and/or HE cells. The method further comprises increasing expression or modifying activity of two, three, four, five, or more hematopoietic genes selected from runx1, spi1, cebpa, tal1, gfi1, gata2 and mllt3 in the endothelial and/or HE cells. By decreasing the expression or activity of the endothelial genes, and by increasing the expression or activity of the hematopoietic genes, formation of HE cells or HSCs (including substantial numbers of LT-HSCs) is stimulated. The decrease in expression or activity of endothelial genes, and the increase in expression or activity of hematopoietic genes, can be directly, for example by administration of inhibitors, transgenes, episomes, mRNA and their derivatives, and/or using gene editing approaches (as described more fully herein). Alternatively, such changes in expression or activity can be induced at least in part indirectly, for example by increasing the expression or activity of DNA (cytosine-5-)-methyltransferase 3 beta (Dnmt3b) and/or GTPase IMAP Family Member 6 (Gimap6). Further, such gene expression modulation can be conducted at least in part by using an agonist of a mechanosensitive receptor or channel (e.g., a Piezo1 agonist), or by applying a cyclic-strain biomechanical stretching to the cells. In various embodiments, at least one or at least two, three, four, five, or more endothelial gene selected from vegfa, hey2, grp116, gnal3, sox17, cdh5, plxnd1, bcl6, and apln are reduced in expression directly or indirectly. In these or other embodiments, at least one or at least two, three, four, five, or more hematopoietic genes selected from runx1, spi1, cebpa, tal1, gfi1, gata2 and mllt3 are increased in expression directly or indirectly.

In some embodiments, expression or activity of the endothelial genes and the hematopoietic genes is modulated at least in part by increasing activity or expression of Dnmt3b and/or Gimap6 in the cells under conditions sufficient for stimulating formation of HE cells or HSCs. In some embodiments, the HE cells are recovered and used for the formation of HSCs. The HSCs can be recovered and optionally expanded for administration to a patient. HSCs are optionally expanded using genetic, pharmacological, or mechanical stimuli described herein.

In some embodiments, the endothelial cells are contacted with an effective amount of an agonist that increases the activity or expression of Dnmt3b, or provides the proper modulation of the endothelial genes and hematopoietic genes. In some embodiments, the agonist is an agonist of a mechanosensitive receptor or a mechanosensitive channel. In some embodiments, the mechanosensitive receptor is Piezo1. Exemplary Piezo1 agonists include Yoda1, Jedi1, and Jedi2. In some embodiments, the effective amount of the Piezo1 agonist (e.g., Yoda1) is in the range of about 0.1 μM to about 300 μM, or about 0.1 μM to about 200 μM, or about 1 μM to about 100 μM, or in some embodiments, about 10 μM to about 100 μM, or about 2.5 μM to about 100 μM or about 2.5 μM to about 50 μM.

Alternative mechanosensitive receptor or mechanosensitive channel agonists (e.g., Piezo1 agonists) can be identified from a chemical library. In these embodiments, an agonist is identified that induces the changes in endothelial and hematopoietic gene expression as described herein. For example, the candidate agonist decreases expression or modifies activity of endothelial genes selected from vegfa, hey2, grp116, gna13, sox17, cdh5, plxnd1, bcl6, and apln in endothelial and/or HE cells; and increases expression or modifies activity of two or more hematopoietic genes selected from runx1, spi1, cebpa, tal1, gfi1, gata2, and mllt3 in endothelial and/or HE cells, upon contact with a candidate compound.

Where the activity or expression of genes is to be increased directly in the endothelial and/or HE cells, various approaches can be employed. For example, mRNA expression of genes can be increased by delivering mRNA transcripts (including modified mRNA) to the cells, or by introducing a transgene and/or an episome, which may have one or more modifications thereto to increase or modify activity. In some embodiments, gene editing is employed to introduce a genetic modification to expression elements in the endothelial or HE cells, such as to increase promoter strength, ribosome binding, or RNA stability. In some embodiments, gene editing is employed to introduce gain-of-function mutations.

In some embodiments, the activity or expression of genes can be decreased directly in the endothelial and/or HE cells. For example, expression or activity of endothelial genes can be reduced by one or more of: introducing a full or partial gene deletion, RNA silencing, antisense oligonucleotide inhibition, and introducing a genetic modification of expression elements (including to decrease promoter strength, ribosome binding, or RNA stability) or introducing a loss-of-function mutation in the endothelial and/or HE cells.

In some embodiments, the invention comprises increasing the activity or expression of Gimap6 in the endothelial and/or HE cells, alone or in combination with Dnmt3b. To increase activity or expression of Gimap6, Gimap6 mRNA transcripts can be introduced to the cells, or alternatively a Gimap6 transgene and/or an episome, and/or introducing a genetic modification of Gimap6 expression elements in the cells (such as one or more modifications to increase promoter strength, ribosome binding, or RNA stability).

In various embodiments, a cell population comprising embryonic bodies, endothelial cells and/or HE cells is introduced to a bioreactor. In some embodiments, the bioreactor provides a cyclic-strain biomechanical stretching to the cells in 2D or 3D culture. For example, the cyclic-strain biomechanical stretching can be applied to a 2D or 3D culture surface. The cyclic-strain biomechanical stretching increases the activity or expression of Dnmt3b and/or Gimap6. For example, a computer controlled vacuum pump system (e.g., the FlexCell™ Tension System, the Cytostretcher System, or similar) attached to a nylon, PDMS, or other biocompatible biomimetic membrane (e.g., of a flexible-bottomed culture plate) can be used to apply circumferential stretch ex vivo to embryonic bodies, endothelial cells or HE cells that contact the membrane in 2D or 3D culture, under defined and controlled cyclic strain conditions.

In various embodiments, the HSC transition is induced by one or more selected from Piezo1 activation; mechanical stretching; introduction of an mRNA, with or without a transgene (i.e., transgene free), an episome, or genetic modification to Dnmt3b; introduction of an mRNA, with or without a transgene (i.e., transgene free), an episome, or genetic modification to Gimap6; introduction of an mRNA, with or without a transgene (i.e., transgene free), an episome, or genetic modification to one or more hematopoietic genes described herein; and introducing a full or partial gene deletion, RNA silencing, antisense oligonucleotide inhibition, or introducing a genetic modification to an endothelial gene(s) described herein.

In some embodiments, the endothelial and/or HE cells are obtained or derived from induced pluripotent stem cells (iPSCs), non-hematopoietic stem cells, or somatic cells such as fibroblasts or endothelial cells. In some embodiments, the endothelial and/or HE cells are obtained or derived from HLA-null cells, HLA-modified cells, gene corrected, viral vector overexpressed, transgene overexpressed, and/or transgene-free cells, or from a genetic induction of embryonic bodies to endothelial cells and/or HE cells. The hemogenic endothelial cells (e.g., Flkl+CD45+ cells, Flkl+CD41+ cells or CD31+CD43+ cells) can be obtained in any manner, including derived from source cells of an allogeneic donor or from the subject to be treated with the HSC (i.e., by chemical, genetic, mRNA, transgene-free, or episome induction of autologous or allogenic cells to hemogenic endothelial cells). In some embodiments, endothelial cells or HE cells are generated from iPSC created using cells from the recipient, off-the-shelf bank, or a universal compatible donor. In some embodiments, developmentally plastic endothelial cells are employed.

In various embodiments, a pharmaceutical composition for cellular therapy is prepared that comprises a population of HSCs prepared by the methods described herein, and a pharmaceutically acceptable vehicle. The pharmaceutical composition may comprise at least about 10² HSCs, or at least about 10³ HSCs, or at least about 10⁴ HSCs, or at least about 10⁵ HSCs, or at least about 10⁶ HSCs, or at least about 10⁷ HSCs, or at least 10⁸ HSCs. In various embodiments, at least about 0.1%, or at least about 1%, or at least about 2%, or at least about 5%, or at least about 10%, or at least about 20%, or at least about 30%, or at least about 40%, or at least about 50% of the HSCs in the composition are LT-HSCs. For example, in some embodiments, the pharmaceutical composition is administered, comprising from about 100,000 to about 4×10⁶ HSCs per kilogram (e.g., about 2×10⁶ cells/kg) of a recipient's body weight.

In some embodiments, a cellular therapy is prepared that comprises a population of HSCs prepared by the methods described herein. In some embodiments, the cellular therapy includes a pharmaceutically acceptable vehicle. The cellular therapy may comprise at least about 10² HSCs, or at least about 10³ HSCs, or at least about 10⁴ HSCs, or at least about 10⁵ HSCs, or at least about 10⁶ HSCs, or at least about 10⁷ HSCs, or at least 10⁸ HSCs. In various embodiments, at least about 0.1%, or at least about 1%, or at least about 2%, or at least about 5%, or at least about 10%, or at least about 20%, or at least about 30%, or at least about 40%, or at least about 50% of the HSCs in the composition are LT-HSCs. For example, in some embodiments, the pharmaceutical composition is administered, comprising from about 100,000 to about 4×10⁶ HSCs per kilogram (e.g., about 2×10⁶ cells/kg) of a recipient's body weight. The number of HSC cells may be modified based on the age and weight of the patient.

The HSCs for transplantation can be generated in some embodiments in a relatively short period of time, such as less than about two months, or less than one about month (e.g., about 4 weeks), or less than about two weeks, or less than about one week, or less than about 6 days, or less than about 5 days, or less than about 4 days, or less than about 3 days. In some embodiments, the developmentally plastic endothelial or HE cells are cultured with modulated activity or expression of the endothelial and hematopoietic genes for 1 to 4 weeks.

HSCs prepared by the methods described herein are administered to a subject (a recipient), e.g., by intravenous infusion or intra-bone marrow transplantation. The methods can be performed following myeloablative, non-myeloablative, or immunotoxin-based (e.g. anti-c-Kit, anti-CD45, etc.) conditioning regimes.

The methods described herein can be used to generate populations of HSC for use in transplantation protocols, e.g., to treat acquired or inherited forms of blood (malignant and non-malignant), bone marrow, metabolic, mitochondrial, and immune diseases. In some embodiments, the HSC populations are derived from autologous cells, e.g., generated from iPSC, which are created using cells from the recipient subject. In some embodiments, the HSC populations are derived from universally compatible donor cells or HLA-null hemogenic endothelial cells or similar cells conducive to become normal HSCs.

These and other aspects and embodiments of the invention are described by the following detailed description of the invention.

### DESCRIPTION OF THE FIGURES

FIG. 1A shows time-lapse confocal imaging of cd41:eGFP⁺ HSCs emerging from flk1:mCherry⁺ endothelial cells in transgenic embryos between 26-42 hpf; the data demonstrates that the silencing of piezo1 attenuates the endothelial-to-HSC transition, whereas pharmacological activation of piezo1 (Yoda1) stimulates HSC formation in control embryos as well as rescues HSC formation in sih-MO embryos. n=5 per group. *P<0.05 vs. control; ^{$}P<0.05 vs. sih-MO.

FIG. 1B is a heat map of differentially expressed genes in E11.5 AGM cells treated with cyclic strain, and Piezo1 activator (Yoda1); indicating that cyclic strain and Piezo1 activation have similar gene expression patterns in AGM during the endothelial-to-hematopoietic transition. n=3 per group.

FIG. 1C shows a graph of hematopoietic colony formation unit (CFU) assays on E11.5 AGM cells, which demonstrates that Yoda1-mediated pharmacological activation of Piezo1 stimulates the endothelial-to-hematopoietic transition. n≥6 per group. *P<0.05 vs. Control. Abbreviations: GEMM (granulocyte, erythroid, macrophage, megakaryocyte); GM (granulocyte macrophage); G (granulocyte); M (macrophage); E (erythroid).

FIG. 1D shows a graph of hematopoietic CFU assays on E11.5 AGM cells, which demonstrate that GsMtX4-mediated pharmacological inhibition of Piezo1 attenuates the inductive impact of cyclic strain on the endothelial-to-HSC transition. n≥6 per group. *P<0.05 vs. Control. Abbreviations: GEMM (granulocyte, erythroid, macrophage, megakaryocyte); GM (granulocyte macrophage); G (granulocyte); M (macrophage); E (erythroid).

FIG. 1E shows hematopoietic CFU assays on E11.5 AGM cells treated with 50 μM Jedi1, 50 μM Jedi2, or 25 μM Yoda1, demonstrating that Jedi1, Jedi2, or Yoda1-mediated Piezo1 activation enhances GEMM formation. N=6 per group. 3.e.e. AGM per each sample. *P<0.05.

FIG. 2A shows an experimental outline (top) and a line graph (bottom). The experimental outline (top) shows a schema representing serial transplantations of HSCs originating in E11.5 mouse AGM followed by treatment with 10% cyclic strain or Yoda1 into myeloablative immunocompromised mice. The line graph (bottom) shows the percentage peripheral blood chimerism from reconstitution of E11.5 AGM (donor; three embryo equivalent)-derived HSCs in a primary transplant (recipient) at four-week intervals between weeks 8-16; indicating that cyclic strain or pharmacological activation of Piezo1 (Yoda1 treatment) to E11.5 AGM stimulates the formation of HSCs. n≥5 primary recipients per group. *P<0.05 vs. control; ^{$}P<0.05 vs. week 8 chimerism. Three embryo equivalent (e.e.) AGM donor cells were injected in each recipient.

FIG. 2B is a graph showing the percentage reconstitution of E11.5 AGM (donor; three embryo equivalent)-derived HSCs to Mac1⁺Gr1⁺ myeloid cells, Cd8⁺Cd3⁺ T-cells, and B220⁺Cd19⁺ B-cells in a primary transplant (recipient) at week 16; indicating that cyclic strain or pharmacological activation of Piezo1 (Yoda1) to E11.5 AGM stimulates the formation of HSCs that reconstitute to the blood. n≥5 primary recipients per group.

FIG. 2C is a line graph showing the percentage peripheral blood chimerism from reconstitution of primary transplant (donor)-derived flow-sorted Lin⁻Sca1⁺c-Kit⁺ HSPCs (n=2000) in a secondary transplant (recipient) at four-week intervals between weeks 8-12; indicating that cyclic strain or Yoda1 treatment of E11.5 AGM produces HSCs that have serial engraftment and self-renewal capacities. n≥5 secondary recipients per group. *P<0.05 vs. control.

FIG. 2D is a graph showing the percentage reconstitution of primary transplant (donor)-derived HSCs to Mac1⁺Gr1⁺ myeloid cells, Cd8⁺Cd3⁺ T-cells, and B220⁺Cd19⁺ B-cells in a secondary transplant (recipient) at week 12; indicating that cyclic strain or Yoda1 treatment of E11.5 AGM produces HSCs that can serially reconstitute to the blood. n≥5 secondary recipients per group.

FIG. 3A shows an experimental outline (top) and a graph (bottom). The experimental outline (top) shows strategies for functional and phenotypic analyses of donor-derived blood lineages in hematopoietic tissues of primary transplant (recipient mice). The graph (bottom) shows the percentage expression of β-major (adult), εγ (embryonic), and β-H1 (embryonic) types of hemoglobin in bone marrow-derived Cd71⁺ Ter119⁺ sorted (donor) erythroid cells; the data indicates that donor HSCs produced following biomechanical stretching or Yoda1-treatment of E11.5 AGM reconstitutes to red cells containing adult hemoglobin. n≥6 per group.

FIG. 3B is a graph showing an overnight culture (O/N) of bone marrow-derived Gr1⁺Mac1⁺ sorted (donor) neutrophils followed by ELISA-based quantification of myeloperoxidase (MPO) proteins; the data demonstrate that donor HSCs were produced following biomechanical stretching or Yoda1 treatment of E11.5 AGM, which reconstitute to functional myeloid cells displaying sufficient MPO levels. n≥5 per group.

FIG. 3C is a graph showing ELISA analyses of pre-immunized immunoglobulin (Ig) isotypes in the peripheral blood of primary transplant (recipient) mice; the data indicates that primary transplant produces B-cells with a complete repertoire of immunoglobulins. n≥6 per group.

FIG. 3D is an image of two gel pictures showing T-cell receptor (TCR_{β}) locus analyses of spleen-sorted Cd3⁺ T cells (donor) (top) or Mac1⁺ myeloid cells (donor; negative control) (bottom); the data indicates that donor HSCs produced T-cells and display T-cell receptor β (TCR β) rearrangement following biomechanical stretching or Yoda1-treatment of E11.5 AGM, which migrate to the spleen and reconstitute to T-cells that possess functional recombination machinery sufficient to rearrange TCR_{β} locus.

FIG. 3E is a dot plot showing delayed-type hypersensitivity assay, which demonstrates that primary transplant (recipient) mice reconstituted with biomechanical stretching or Yoda1-treated E11.5 AGM-derived donor HSCs possess T-cell mediated immune response. n≥6 per group. *P<0.05 vs. right footpad (negative control).

FIG. 4 shows Venn diagrams of genes up-regulated in E11.5 AGM cells treated with cyclic strain and/or Yoda1 in the context of genes up-regulated during EC vs. HSC (\textcircled{1}), EC vs. HEC (\textcircled{2}), and HEC vs. HSC (\textcircled{3}). The Venn comparison of the commonly upregulated genes in the above analyses (\textcircled{1} vs. \textcircled{2} vs. \textcircled{3}) demonstrates that both circumferential stretching and Piezo1 activation specifically stimulate Dnmt3b transcript expression and Gimap6 transcript expression during the endothelial-to-HSC transition.

FIG. 5A shows two graphs of the protein levels of Dnmt3b and Dnmt3a in nuclear fractions of E11.5 mouse AGM cells treated with cyclic strain or Yoda1; the data demonstrates that circumferential stretching or Piezo1 activation specifically stimulates Dnmt3b protein expression levels without impacting the expression of Dnmt3a. n≥3 per group. *P<0.05 vs. Control.

FIG. 5B shows a graph of the hematopoietic CFU assays of E11.5 mouse AGM cells treated with cyclic strain or Yoda1 in the presence of Nanaomycin (Nana); the data indicates that the pharmacological inhibition of Dnmt3b attenuates the endothelial-to-HSC transition stimulated by circumferential stretch or Piezo1 activation. n≥6 embryos per group. *P<0.05 vs. Control; ^{$}P<0.05 vs. Stretch; ⁺P<0.05 vs. Yoda1.

FIG. 5C is a graph showing the results of time-lapse confocal imaging of cd41:eGFP⁺ HSCs emerging from flk1:mCherry⁺ endothelial cells in transgenic embryos between 26-42 hpf; the data demonstrates that the silencing of dnmt3bb.1 attenuates the endothelial-to-HSC transition stimulated by piezo1 activation, and the specificity of Nanaomycin for Dnmt3b over Dnmt3a. n≥5 per group. *P<0.05 vs. control; ^{$}P<0.05 vs. Yoda1.

FIG. 6A shows differential expression of endothelial and hematopoietic genes in cyclic stretch- or Yoda1-treated E11.5 AGM cells, indicating that cyclic strain and Piezo1 activation repress the expression of endothelial genes while stimulating the expression of hematopoietic genes during the endothelial-to-hematopoietic transition.

FIG. 6B shows qRT-PCR analysis of endothelial and hematopoietic gene expression; demonstrating that cyclic strain or Piezo1 activation represses the expression of endothelial genes and stimulates the expression of hematopoietic genes in E11.5 AGM cells. n=5 per group. *P≤0.05 vs. Control.

FIG. 6C shows hematopoietic CFU assays of human iPSC-derived MACS-sorted CD34+ cells following Yoda1-treatment; this demonstrates that the pharmacological activation of PIEZO1 stimulates multipotent GEMM progenitor formation and human hematopoiesis. n=6 per group; 20,000 hCD34+ cells per sample. *≤0.05 vs. Control.

FIG. 6D shows percentage of bone marrow chimerism (left) after 8-10 weeks from the injection of human PSC (DF19-9-7T)-derived hCD34+ hematopoietic cells in humanized mice (primary transplant), indicating that Yoda1-mediated pharmacological activation of Piezo1 enhances the formation of engraftable hCD34+ cells. FIG. 6D further shows the percentage of bone marrow reconstitution of human PSC-derived hCD34+ hematopoietic cells (right) to human CD33+ myeloid cells, human CD3+ T cells, and human CD19+ B-cells, indicating that pharmacological activation of Piezo1 (Yoda1) stimulates the formation of hCD34+ hematopoietic cells that reconstitute multi-lineage blood. n=8 (control; for primary transplant) and n=8 (Yoda1 treatment, primary transplant). ***P≤0.001.

FIG. 6E shows percentage of peripheral blood chimerism after 16 weeks from the injection of bone marrow cells derived from primary transplants of hCD34+ hematopoietic cells derived from DFT19-9-7T hPSC line, in humanized mice (secondary transplant) (left), indicating that Yoda1-mediated pharmacological activation of Piezo1 enhances the formation of self-renewing LT-HSCs. FIG. 6E further shows the percentage peripheral blood reconstitution of primary transplant bone marrow-derived human hematopoietic cells in secondary transplant (right) to human CD33+ myeloid cells, human CD3+ T cells, and human CD19+ B cells, indicating that pharmacological activation of Piezo1 (Yoda1) stimulates the formation of human LT-HSCs that reconstitute multi-lineage blood upon serial transplantation. N=5 (Yoda1 treatment, secondary transplant).

FIG. 7A demonstrates with FACS analyses that cyclic stretch promotes the EC to HEC transition (left) as well as HEC to HSPC transition (center). Analysis of HSPC expansion with cyclic stretch is shown on the right. N=6. **P≤0.001, *P≤0.05. FIG. 7A (top) illustrates the application of 10% cyclic stretch on mouse E11.5 AGM-sorted ECs (CD31⁺), HECs (CD31⁺cKit⁺) and HSPCs (cKit⁺) cells followed by FACS analysis.

FIG. 7B shows FACS analyses of human-derived embryoid bodies (EBs) at day 8 of hematopoietic differentiation, indicating that Yoda1-mediated PIEZO1 activation enhances the formation of hCD43^{neg}CD235^{neg}CD144⁺CD34⁺ HECs in control but not in PIEZO1^{-/-} PSCs. The loss of PIEZO1 did not influence HEC formation. N=3 per group. **P≤0.001 *P≤0.05.

FIG. 7C is a FACS plot of CD34⁺CD90⁺ HSCs, derived at day 8+7 of hematopoietic differentiation of human PSCs, indicating that Yoda1-mediated PIEZO1 activation enhances the formation of HSCs from hPSC.

### DETAILED DESCRIPTION

During fetal development, a subset of endothelial cells in the aorta-gonad-mesonephros (AGM) are hemogenic endothelial cells, which change their fate to become HSCs that ultimately colonize the fetal liver and bone marrow. However, the identities of the factors stimulating hemogenic endothelial cells remain elusive, limiting the utility of hemogenic endothelial cells as a potential source of functional HSCs. Blood flow-mediated shear-stress on the endothelial lining stimulates the endothelial emergence of HSCs. However, using Cdh5-null zebrafish and murine models, it was established that functional HSCs emerge despite early circulation arrest. Anderson H, et al., “Hematopoietic stem cells develop in the absence of endothelial cadherin 5 expression.” Blood 2015. These cdh5-silenced models were used in accordance with this disclosure as a pivot to study shear-stress and/or nitric oxide synthase (NOS)-independent biomechanical forces triggering functional HSC emergence, to investigate additional mechanisms by which pulse-pressure-mediated circumferential stretch governs HSC emergence.

Attempts to generate HSCs from hemogenic endothelial cells in the laboratory have been largely unsuccessful, in part due to a lack of knowledge about factors that stimulate HSC emergence from hemogenic endothelial cells. It is now established that circumferential vascular stretch due to pulsations from a beating heart triggers functional HSCs to emerge from hemogenic endothelial cells, which can ultimately engraft and differentiate into definitive lineages. In addition, the activation of stretch-sensitive transient receptor potential cation channel-subfamily vanilloid member 4 (Trpv4) channels rescued HSC formation in silent heart (tnnt2; sih)-silenced embryos in the absence of heartbeat and blood flow. See WO 2017/096215, which is hereby incorporated by reference in its entirety.

The present disclosure is based at least in part on the discovery that biomechanical and/or pharmacological activation of a mechanosensitive receptor (e.g., Piezo1) enhances Dnmt3b expression for hematopoietic stem cell (HSC) formation, which in turn modulates the expression of a core set endothelial genes and hematopoietic genes and their regulators. As demonstrated herein, cdh5-morphant (cdh5-MO) embryos have a heartbeat-mediated pulsation in blood vessels without cardiac output and active blood flow. Pulsation-derived stretching activates Piezo1 mechanosensitive channels that further enhance Dnmt3b expression in the AGM to stimulate the endothelial-to-HSC transition. The simulation of pulsation or the pharmacological activation of Piezo1 also yields at least three times higher amounts of LT-HSCs, which reconstitute to normal, functional multi-lineage adult blood upon serial transplantation.

Accordingly, the results of the present disclosure demonstrate how heartbeat-mediated biomechanical forces stimulate cell-fate transitions and stem cell formation by activating mechanosensitive channels as well as epigenetic machinery. The development, expansion, and stemness maintenance of LT-HSCs are major challenges in HSC transplantation and cellular therapies for treating blood and bone marrow diseases. The present disclosure provides genetic and pharmacological targets to develop LT-HSCs. In the various aspects, this disclosure provides genetic, pharmacological, and mechanical stimuli for transitioning endothelial cells to hemogenic endothelial (HE) cells, and for transitioning HE cells to HSCs, including HSCs that comprise a significant level of LT-HSCs. The disclosure further provides methods for expanding HSCs using the genetic, pharmacological, and mechanical stimuli.

In one aspect, the invention provides a method of preparing a population HSCs comprising LT-HSCs. In certain embodiments, the method comprises providing a cell population comprising endothelial and/or HE cells, and decreasing expression or modifying activity of two or more endothelial genes selected from vegfa, hey2, grp116, gna13, sox17, cdh5, plxnd1, bcl6, and apln in the endothelial and/or HE cells. The method further comprises increasing expression or modifying activity of two or more hematopoietic genes selected from runx1, spi1, cebpa, tal1, gfi1, gata2 and mllt3 in the endothelial and/or HE cells, so as to stimulate formation of the HSCs including LT-HSCs.

In some embodiments, the method comprises decreasing expression or modifying activity of three or five or more endothelial genes selected from vegfa, hey2, grp116, gna13, sox17, cdh5, plxnd1, bcl6, and apln in the endothelial and/or HE cells; and increasing expression or modifying activity of three or five or more hematopoietic genes selected from runx1, spi1, cebpa, tal1, gfi1, gata2, and mllt3 in the endothelial and/or HE cells. For example, in some embodiments the method comprises decreasing expression or modifying activity of vegfa, hey2, grp116, gna13, cdh5, and plxnd1 in the endothelial and/or HE cells; and increasing expression or modifying activity of runx1, spi1, cebpa, tal1, and gata2 in the endothelial and/or HE cells.

In some embodiments, the expression or activity of at least one, two, three, or five hematopoietic genes are increased directly. As used herein, the expression or activity of a gene is increased “directly”, where a nucleic acid encoding a functional copy of said gene is introduced to the cell or where modifications are made to the endogenous gene that increase its expression or relevant activity. For example, activity or expression can be increased directly using approaches independently selected from: introducing an encoding mRNA, introducing an encoding transgene or an episome, introducing a genetic modification of expression elements, and introducing gain-of-function mutation(s). In some embodiments, the full set of hematopoietic genes increased in expression or activity are increased in expression or activity directly.

In accordance with various embodiments of the disclosure employing expression of factors using an episome, such embodiments may comprise introducing a non-integrating episomal plasmid expressing the desired factors, i.e., for the creation of transgene-free and virus-free cell population. Known episomal plasmids can be employed with limited replication capabilities and which are therefore lost over several cell generations.

In some embodiments, the expression or activity of at least one, two, three, or five endothelial genes are decreased directly. As used herein, the expression or activity of a gene is decreased directly, where a nucleic acid or pharmacological inhibitor are introduced to the cell, or where modifications are made to the endogenous gene that decrease its expression or relevant activity. For example, activity or expression can be decreased directly using approaches independently selected from: introducing a full or partial gene deletion, RNA silencing, antisense oligonucleotide inhibition, pharmacological inhibition, and introducing a genetic modification of expression elements or introducing loss-of-function mutation(s). In some embodiments, the full set of endothelial genes decreased in expression or activity in a certain embodiment, are decreased in expression or activity directly.

In some embodiments, the expression or activity of one or more endothelial genes and one or hematopoietic genes can be modulated indirectly, for example, by increasing activity or expression of DNA (cytosine-5-)-methyltransferase 3 beta (Dnmt3b) and/or GTPase IMAP Family Member 6 (Gimap6) in the endothelial cells under conditions (including expression level and duration of higher expression) sufficient for stimulating formation of HSCs.

Dnmt3b (DNA (cytosine-5-)-methyltransferase 3 beta) is a DNA methyltransferase. Dnmt3b that localizes primarily to the nucleus and its expression is developmentally regulated. Gimap6 is a member of the GTPases of immunity-associated proteins (GIMAP) family. GIMAP proteins contain GTP-binding and coiled-coil motifs.

In some embodiments, the endothelial cells or HE cells, or HSCs according to the various embodiments are contacted with an effective amount of an agonist of a mechanosensitive receptor or a mechanosensitive channel that increases the activity or expression of Dnmt3b, and thereby indirectly modulating the levels of expression or modifying activity of the endothelial and hematopoietic genes. In some embodiments, the mechanosensitive receptor is Piezo1. An exemplary Piezo1 agonist is Yoda1. Other exemplary Piezo1 agonists include Jedi1 and Jedi2.

Yoda1 (2-[5-[[(2,6-Dichlorophenyl)methyl]thio]-1,3,4-thiadiazol-2-yl]-pyrazine) is a small molecule agonist developed for the mechanosensitive ion channel Piezo1. Syeda R, “Chemical activation of the mechanotransduction channel Piezo1.” eLife (2015). Yoda1 has the following structure:

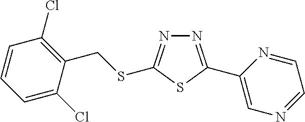 [SMILES: Clc1cccc(Cl)c1CSc1nnc(-c2cnccn2)s1]

Derivatives of Yoda1 can be employed in various embodiments. For example, derivatives comprising a 2,6-dichlorophenyl core are employed in some embodiments. Exemplary agonists are disclosed in Evans E L, et al., “Yoda1 analogue (Dooku1) which antagonizes Yoda1-evoked activation of Piezo1 and aortic relaxation,” British J. of Pharmacology 175(1744-1759): 2018. Jedi1 and Jedi2 are described in Wang Y., et al., “A lever-like transduction pathway for long-distance chemical- and mechano-gating of the mechanosensitive Piezo1 channel,” Nature Communications (2018)9:1300. Jedi1 and Jedi2 have a 3-carboxylic acid methylfuran structural motif. Other Piezo1 agonists sharing this motif may be employed in accordance with embodiments of the invention.

In some embodiments, the effective amount of the Piezo1 agonist (e.g., Yoda1, Jedi1, or Jedi2) is in the range of about 0.1 μM to about 500 μM, or about 0.1 μM to about 300 μM, or about 0.1 μM to about 200 μM, or about 0.1 μM to about 100 μM, or in some embodiments, about 1 μM to about 300 μM, about 1 μM to about 200 μM, about 1 μM to about 100 μM, about 1 μM to about 50 μM, or about 10 μM to about 100 μM, or about 10 μM to about 100 μM or about 10 μM to about 50 μM.

Alternative agonists, including of Piezo1, can be identified in a chemical library. Such chemical library can comprise compounds that bind and/or activate Piezo1, or other mechanosensitive receptor or channel. The library may comprise derivatives of Yoda1, which may optionally have a 2,6-dichlorophenyl core, or a chemical mimetic thereof. The library may comprise or further comprise derivatives of Jedi1 and/or Jedi2, which may optionally comprise a furan core (e.g., 3-carboxylic acid methylfuran core, or derivatives or chemical mimetic thereof). The library can be screened for compounds that decrease expression or activity of the endothelial genes described herein in endothelial and/or HE cells; and which increase expression or activity of the hematopoietic cells described herein in endothelial and/or HE cells, upon contact with the candidate compound. Changes in expression or activity can be determined by comparison to control cells, i.e., cells that are not contacted with the candidate compound. In some embodiments, cells contacted with Yoda1, Jedi2, and/or Jedi2 can be used as a positive control for gene expression modulations.

In these embodiments, the invention can provide a method for making hematopoietic stem cells (HSCs), which comprises: contacting a panel of chemical compounds with endothelial cells and/or hemogenic endothelial cells, and determining a change in expression level induced by said chemical compounds of: Dnmt3b or Gimap6; at least two (or at least three or at least five) of vegfa, hey2, grp116, gna13, sox17, cdh5, plxnd1, bcl6, and apln; and at least two (or at least three or at least five) of runx1, spi1, cebpa, tal1, gfi1, gata2, and mllt3. A compound is then selected that induces one or more of the following changes in gene expression: increase in expression of Dnmt3b and/or Gimap6; decrease in expression of three of more of vegfa, hey2, grp116, gna13, sox17, cdh5, plxnd1, bcl6, and apln; and increase in expression of two or more runx1, spi1, cebpa, tal1, gfi1, gata2, and mllt3. The selected compound can then be used (e.g., in a bioreactor) to induce the transition of endothelial cells and/or hemogenic endothelial cells to HSCs. The resulting HSCs are self-renewing HSCs that can engraft and reconstitute multi-lineage blood. In some embodiments, the selected compound decreases expression of vegfa, hey2, grp116, gna13, cdh5, and plxnd1 in the endothelial and/or HE cells; and increases expression of runx1, spi1, cebpa, tal1, and gata2 in the endothelial and/or HE cells.

In some embodiments, the activity or expression of Dnmt3b can be increased directly in the endothelial or HE cells. For example, mRNA expression of Dnmt3b can be increased by delivering Dnmt3b-encoding transcripts to the cells, or by introducing a Dnmt3b-encoding transgene, or a transgene-free method, not limited to introducing an episome to the cells, which may have one or more nucleotide modifications (or encoded amino acid modifications) thereto to increase or modify activity. In some embodiments, gene editing is employed to introduce a genetic modification to Dnmt3b expression elements in the endothelial cells, such as to increase promoter strength, ribosome binding, RNA stability, or impact RNA splicing. In some embodiments, a gain-of-function mutation is introduced in the Dnmt3b gene.

In some embodiments, the invention comprises increasing the activity or expression of Gimap6 in the endothelial cells, alone or in combination with Dnmt3b and/or other modified genes upon cyclic strain or Piezo1 activation. To increase activity or expression of Gimap6, Gimap6-encoding mRNA transcripts can be introduced to the cells, transgene-free approaches can also be employed, including but not limited, to introducing an episome to the cells; or alternatively a Gimap6-encoding transgene, which may have one or more nucleotide modifications (or encoded amino acid modifications) thereto to increase or modify activity. In some embodiments, gene editing is employed to introduce a genetic modification to Gimap6 expression elements in the endothelial cells (such as one or more modifications to increase promoter strength, ribosome binding, RNA stability, or to impact RNA splicing). In some embodiments, a gain-of-function mutation is introduced in the Gimap6 gene.

In some embodiments, mRNA and/or episome(s) (e.g., encoding Dnmt3b or Gimap6, or encoding one or more the hematopoietic genes described herein) is produced synthetically, such as by direct chemical synthesis or in vitro transcription, and introduced into endothelial cells. Known chemical modifications can be used to avoid the innate-immune response in the cells. For example, synthetic RNA comprising only canonical nucleotides can bind to pattern recognition receptors, and can trigger a potent immune response in cells. This response can result in translation block, the secretion of inflammatory cytokines, and cell death. RNA comprising certain non-canonical nucleotides can evade detection by the innate immune system, and can be translated at high efficiency into protein. See U.S. Pat. No. 9,181,319, which is hereby incorporated by reference, particularly with regard to nucleotide modification to avoid an innate immune response. mRNA can be introduced into the cells by known methods once or periodically during HSC production.

In some embodiments, expression of Dnmt3b and/or Gimap6 and/or one or more hematopoietic genes described herein is increased by introducing a transgene into the cells, which can direct a desired level of overexpression (with various promoter strengths or other selection of expression control elements). Transgenes can be introduced using various viral vectors or transfection reagents known in the art. In some embodiments, expression of Dnmt3b and/or Gimap6 and/or hematopoietic genes is increased by a transgene-free method (e.g., episome delivery).

In some embodiments, expression or activity of genes are modulated using a gene editing technology, for example, to introduce one or more modifications to alter promoter strength, ribosome binding, RNA stability, or RNA splicing. Various editing technologies are known, and include CRISPR, zinc fingers (ZFs) and transcription activator-like effectors (TALENs). Fusion proteins containing one or more of these DNA-binding domains and the cleavage domain of Fokl endonuclease can be used to create a double-strand break in a desired region of DNA in a cell (See, e.g., US Patent Appl. Pub. No. US 2012/0064620, US Patent Appl. Pub. No. US 2011/0239315, U.S. Pat. No. 8,470,973, US Patent Appl. Pub. No. US 2013/0217119, U.S. Pat. No. 8,420,782, US Patent Appl. Pub. No. US 2011/0301073, US Patent Appl. Pub. No. US 2011/0145940, U.S. Pat. Nos. 8,450,471, 8,440,431, 8,440,432, and US Patent Appl. Pub. No. 2013/0122581, the contents of all of which are hereby incorporated by reference). In some embodiments, gene editing is conducting using CRISPR associated Cas system, as known in the art. See, for example, U.S. Pat. Nos. 8,697,359, 8,906,616, and 8,999,641, which is hereby incorporated by reference in its entirety.

In various embodiments, a cell population comprising developmentally plastic endothelial or HE cells (including but not limited to embryonic bodies) is introduced to a bioreactor. In some embodiments, the bioreactor provides a cyclic-strain biomechanical stretching, as described in WO 2017/096215, which is hereby incorporated by reference in its entirety. The cyclic-strain biomechanical stretching increases the activity or expression of Dnmt3b and/or Gimap6, which in turn reduces expression of the endothelial genes described herein, and increased expression of the hematopoietic genes described herein. In these embodiments, mechanical means apply stretching forces to the cells in 2D or 3D culture. For example, a computer controlled vacuum pump system (e.g., the FlexCell™ Tension System, the Cytostretcher System, or similar) can be attached to a nylon, PDMS, or other biocompatible or biomimetic membrane that is employed as a culture surface. The system can then be used to apply circumferential stretch ex vivo to the cells in 2D or 3D culture, under defined and controlled cyclic strain conditions.

In some embodiments, the cyclic-strain biomechanical stretching decreases expression or activity of the endothelial genes in the endothelial and/or HE cells; and increases expression or activity of the hematopoietic genes in the endothelial and/or HE cells, so as to stimulate formation of the HSCs.

In various embodiments, the HSC transition is induced by at least means selected from Piezo1 activation, mechanical stretching, introduction of an mRNA, transgene, transgene-free (e.g., episome), or genetic modification to Dnmt3b, and/or introduction of an mRNA, transgene, transgene-free (e.g., episome), or genetic modification to Gimap6. In various embodiments, at least one hematopoietic gene described herein is directly increased in expression or activity, and/or at least one endothelial gene described herein is directly decreased in expression or activity in the endothelial or HE cells.

The endothelial cells or HE cells can be obtained or derived from a subject who has a blood, bone marrow, metabolic, or immune disease. In some embodiments, the subject does not have a hematological malignancy. The population of HSCs can be administered to a recipient. For autologous HSC transplantation, source cells for iPS cells, endothelial cells and/or HE cells will have been derived from the recipient.

In some embodiments, the endothelial cells and/or HE cells are obtained or derived from induced pluripotent stem cells (iPSCs), non-hematopoietic stem cells, or somatic cells, including but not limited to fibroblasts and endothelial cells. In some embodiments, the endothelial or HE cells are obtained or derived from HLA-null cells, HLA-modified cells, and/or transgene-free cells, or from a genetic induction of endothelial cells to HE cells. The hemogenic endothelial cells (e.g., Flkl+CD45+ cells, Flkl+CD41+ cells or CD31+CD43+ cells) can be obtained in any manner, including from source cells from an allogeneic donor or from the subject to be treated with the HSC. For example, HE cells may be obtained by chemical, genetic, transgene-free, or episome induction of autologous or allogenic cells to hemogenic endothelial cells. In some embodiments, HE cells are generated from iPSC created from cells of the recipient, or from cells that are HLA-modified, or from cells that are HLA-null cells. In some embodiments, the HE cells are obtained or derived from cells of a subject, wherein the subject is a universally compatible donor. Methods for preparing hemogenic endothelial cells are known in the art, and include generation from human pluripotent stem cells. See, WO 2017/096215 and US 2019/0119643, which are hereby incorporated by reference in their entireties. See also, Ditadi et al., Nature Cell Biol. 17(5) 580-591 (2015); Sugimura et al., Nature 2017; 545(7655):432-438; Nakajima-Takagi et al, Blood. 2013; 121(3):447-458; Zambidis et al., Blood. 2008 Nov. 1; 112(9):3601-14 and Park et al, Cytometry A. 2013 January; 83(1): 114-126 (human embryoid body (hEB)-based hemato-endothelial differentiation methods for efficient hiPSC differentiation); Choi et al., Cell Rep. 2012 Sep. 27; 2(3): 553-567 (hPSC differentiation in coculture with OP9); Sandler et al, 2014 Jul. 17; 511(17509):312-318 (endothelial cells to hematopoietic cells); see also Sluvkin, Blood 2013 122:4035-4046. In some embodiments, the number of HE cells to initiate the production of HSCs is at least about 10² cells, about 10³ cells, about 10⁴ cells, about 10⁵ cells, about 10⁶ cells, about 10⁷ cells, or at least 10⁸ cells. In some embodiments, the hematopoietic stem cells produced according to this disclosure comprise long term hematopoietic stem cells (LT-HSCs), which exhibit superior engraftment, and reconstitute to functional, multi-lineage adult blood in the recipient. In some embodiments, HSCs include CD34+ cells.

In some embodiments, the pluripotent stem cells are induced pluripotent stem cells (iPSCs) prepared by reprogramming somatic cells. For example, somatic cells may be reprogrammed by expression of reprogramming factors selected from Sox2, Oct3/4, c-Myc, Nanog, Lin28, and klf4. In some embodiments, the reprogramming factors are Sox2, Oct3/4, c-Myc, Nanog, Lin28, and klf4. In some embodiments, the reprogramming factors are Sox2, Oct3/4, c-Myc, and klf4. Methods for preparing iPSCs are described, for example, in U.S. Pat. Nos. 10,676,165; 9,580,689; and 9,376,664, which are hereby incorporated by reference in their entireties. In various embodiments, reprogramming factors are expressed using well known viral vector systems, such as lentiviral or Sendai viral systems. Alternatively, reprogramming factors can be expressed by introducing mRNA(s) encoding the reprogramming factors into the somatic cells. Further still, iPSCs may be created by introducing a non-integrating episomal plasmid expressing the reprogramming factors, i.e., for the creation of transgene-free and virus-free iPSCs. Known episomal plasmids can be employed with limited replication capabilities and which are therefore lost over several cell generations. In some embodiments, iPSCs are generated from somatic cells such as (but not limited to) fibroblasts or PBMCs. In various embodiments, the iPSCs are autologous or allogenic (e.g., HLA-matched) with respect to a recipient. In some embodiments, iPSCs are HLA-modified or HLA-null cells.

In various embodiments, the HSCs generated are expanded. For example, the HSCs can be expanded according to methods disclosed in U.S. Pat. Nos. 8,168,428; 9,028,811; 10,272,110; and 10,278,990, which are hereby incorporated by reference in their entireties. For example, in some embodiments, ex vivo expansion of HSCs employs prostaglandin E2 (PGE2) or a PGE2 derivative.

In various embodiments, a pharmaceutical composition for cellular therapy is prepared that comprises a population of HSCs prepared by the methods described herein, and a pharmaceutically acceptable vehicle. The pharmaceutical composition may comprise at least about 10² HSCs, or at least about 10³ HSCs, or at least about 10⁴ HSCs, or at least about 10⁵ HSCs, or at least about 10⁶ HSCs, or at least about 10⁷ HSCs, or at least about 10⁸ HSCs. In some embodiments, subpopulations of cells (e.g., LT-HSCs) can be isolated or enriched using, for example, cell sorting approaches. In various embodiments, at least about 0.1%, or at least about 0.5%, or at least about 1%, or at least about 2%, or at least about 3%, or at least about 5%, or at least about 10%, or at least about 20%, or at least about 30%, or at least about 40%, or at least about 50% of the HSCs in the composition are LT-HSCs. In various embodiments, the composition comprises from about 2 to about 25% LT-HSCs, and will comprise from about 5% to about 25% in some embodiments. For example, in some embodiments, the pharmaceutical composition is administered, comprising from about 100,000 to about 4×10⁶ (CD34+) HSCs per kilogram (e.g., about 2×10⁶ cells/kg) of a recipient's body weight. In some embodiments, the pharmaceutical composition comprises at least about 10³, at least about 10⁴, or at least about 10⁵ LT-HSC cells.

The HSCs for therapy or transplantation can be generated in some embodiments in a relatively short period of time, such as less than two months, or less than one month, or less than about two weeks, or less than about one week, or less than about 6 days, or less than about 5 days, or less than about 4 days, or less than about 3 days. In some embodiments, the endothelial cells are cultured with increased Dnmt3b and/or Gimap6 activity or expression for 1 to 4 weeks.

The cell composition may further comprise a pharmaceutically acceptable carrier or vehicle suitable for intravenous infusion or other administration route, and may include a suitable cryoprotectant. An exemplary carrier is DMSO (e.g., about 10% DMSO). Cell compositions may be provided in unit vials or bags, and stored frozen until use. In certain embodiments, the volume of the composition is from about one fluid ounce to one pint.

HSCs generated using the methods described herein are administered to a subject (a recipient), e.g., by intravenous infusion or intra-bone marrow transplantation. The methods can be performed following myeloablative, non-myeloablative, or immunotoxin-based (e.g. anti-c-Kit, anti-CD45, etc.) conditioning regimes.

The methods described herein can be used to generate populations of HSC for use in transplantation protocols, e.g., to treat blood (malignant and non-malignant), bone marrow, metabolic, and immune diseases. In some embodiments, the HSC populations are derived from autologous cells or universally-compatible donor cells or HLA-modified or HLA null cells. That is, HSC populations are generated from HE cells, the HE cells derived from developmentally plastic endothelial cells or iPSCs that were prepared from cells of the recipient subject or prepared from donor cells (e.g., universal donor cells, HLA-matched cells, HLA-modified cells, or HLA-null cells). In some embodiments, autologous-derived cells are used, and the recipient subject has a condition selected from multiple myeloma; non-Hodgkin lymphoma; Hodgkin disease; acute myeloid leukemia; neuroblastoma; Germ cell tumors; autoimmune disorders (systemic lupus erythematosus (SLE), systemic sclerosis); myelodysplastic syndrome, amyloidosis; or other condition treatable using an autologous HSC transplant. In some embodiments, autologous-derived cells (e.g., HSC are generated from cells from the recipient subject) are used, and the recipient subject does not have a hematological malignancy.

In some embodiments, the recipient subject has a condition selected from Acute myeloid leukemia; Acute lymphoblastic leukemia; Chronic myeloid leukemia; Chronic lymphocytic leukemia; Myeloproliferative disorders; Myelodysplastic syndromes; Multiple myeloma; Non-Hodgkin lymphoma; Hodgkin disease; Aplastic anemia; Pure red-cell aplasia; Paroxysmal nocturnal hemoglobinuria; Fanconi anemia; Thalassemia major; Sickle cell anemia; Severe combined immunodeficiency (SCID); Wiskott-Aldrich syndrome; Hemophagocytic lymphohistiocytosis; Inborn errors of metabolism;

Epidermolysis bullosa; Severe congenital neutropenia; Shwachman-Diamond syndrome; Diamond-Blackfan anemia; Pearson Syndrome, and Leukocyte adhesion deficiency. In some such embodiments, allogeneic-derived or universally-compatible donor cells or HLA-modified or HLA-null cells are used for generating the HE cells. For example, HSC are generated from cells from a donor subject, that is, a subject other than the recipient subject. In some embodiments, the donor subject is matched with the recipient subject based on blood type and Human leukocyte antigen (HLA) typing).

As used herein, the term “about” means±10% of the associated numerical value.

These and other aspects of the invention will now be described with the following non-limiting Examples.

### Examples

During definitive hematopoiesis, the first set of HSCs are born from hemogenic endothelial cells in the AGM during fetal development. Therefore, endothelial and/or hemogenic endothelial cells could be a source for developing or expanding HSCs for clinical use provided the establishment of a repertoire of intrinsic and extrinsic factors that exist in the AGM microenvironment.

Once formed, AGM-derived HSCs migrate to the fetal liver and bone marrow, where they undergo asymmetric division into long-term (LT) and short-term (ST) HSCs. While LT HSCs preserve a pool of HSCs by further undergoing asymmetric division, ST-HSCs support the dynamic demands of blood production by symmetric division. The induction of seven transcription factors (ERG, HOXA5, HOXA9, HOXA10, LCOR, RUNX1, and SPI1) (Sugimura, R, et al., Nature, 2017) as well as the induction of FGRS (Fosb, Gfi1, Runx1, and Spi1) transcription factors, coupled with vascular niche-derived angiocrine cytokines (TGFβ, CXCR7, CXCR4, and BMP) (Lis, R. et al., Nature, 2017), enhance the endothelial-to-hematopoietic transition. However, these approaches do not endow endothelial or hemogenic endothelial cells with LT-HSC function and properties. Further, since many of these transcription factors are associated with hematopoietic malignancies when deregulated, their overexpression-based studies, using integrating vectors or transgene-free approaches, do not allow for analysis of gene dosage effects. Moreover, ex vivo expanded HSCs do not eliminate the need to find HLA-matched healthy donor HSCs. Therefore, it is critical to analyze cell-extrinsic or non-integrating factors contributing to de novo LT-HSC formation from hemogenic endothelial cells in order to develop an autologous or off-the-shelf reservoir of LT-HSCs for life-long blood formation.

The process of endothelial cell fate transitioning to HSC is characterized by an early loss of endothelial potential, along with a gradual unfolding of the hematopoietic program. There may exist epigenetic mechanism(s) imparting long-term silencing of endothelial gene(s) during the EHT. While EZH1 actively represses the definitive hematopoietic program during the primitive hematopoiesis period (Vo, L T, et al., Nature, 2018), ISWI chromatin remodeling regulates both primitive and definitive hematopoiesis (Huang H T, et al., Nat. Cell. Biol., 2013). Further, despite dnmt3b regulating c-myb expression for HSPC maintenance (Gore A V, et al., Elife, 2016), the role of dnmt3b in endothelial gene silencing or in de novo LT-HSC formation is unknown. It is unknown which mechanisms could permanently modify the endothelial epigenetic landscape to support formation of LT-HSCs.

As disclosed herein, the present disclosure demonstrates how heartbeat and/or pulsation-mediated biomechanical stretching and/or pharmacological activation of the Piezo1 mechanosensitive pathway impacts expression of core genes to erase the endothelial epigenetic landscape to form HSCs (including LT-HSCs). Furthermore, a bioreactor was developed that mimics pulsation-like conditions and established Piezo1 as a pharmacological target to stimulate and scale-up LT-HSC formation.

### Heartbeat-Mediated Pulsation Stimulates the Endothelial-to-HSC Transition.

An unbiased zebrafish ethylnitrosourea (ENU) mutagenesis screen yielded malbec (bw209^{mlb}), a zebrafish mutant for cadherin-5 (cdh5, ve-cdh). malbec and cdh5-morphant (MO) embryos display normal primitive and definitive hematopoiesis despite circulatory defects.

To identify blood flow and shear stress-independent biomechanical forces that stimulate the endothelial-to-HSC transition, the function and anatomy of the heart was analyzed as well as blood vessels in cdh5-deficient embryos.

Microangiography was first performed by injecting fluorescent dextran beads in the atrium of the two-chamber heart of the zebrafish embryo, and the dextran beads were then tracked in circulation. While the fluorescent dextran beads passed through the atrioventricular (AV) valve and the ventricle to enter general circulation in control embryos, they were trapped in the atrium of cdh5-morphant embryos.

To examine the structure of the heart, hearts were isolated from the control and cdh5-silenced embryos and immunohistochemistry was performed for the endothelial lining (gfp) and cardio-myocytes (mf20). It was found that the atrium (A), atrioventricular (AV) valve, ventricle (V), and outflow tract (OT) were formed in cdh5-morphants, but the AV valve was elongated and distorted.

To investigate why circulation was impaired in the cdh5-silenced embryos, the vascular structure was analyzed, as well as the blood circulation, heart rate, cardiac output, and cardiac tamponade in the cdh5-silenced embryos.

The integrity of the endothelial lining was analyzed in mlb×kdr:dsRED embryos. It was found that the structure of both arteries and veins were intact in cdh5-deficient embryos.

The temporal development of the heart, heartbeat, blood vessels, blood circulation, and HSC formation are conserved in zebrafish, mouse, and man. During zebrafish development, the heart begins to beat around 23 hours post fertilization (hpf), the blood circulation begins at approximately 24-26 hpf, and definitive HSCs emerge from hemogenic endothelial cells in the AGM region between 30-48 hpf.

To analyze the circulation in blood vessels before and after the heart begins to beat, time-lapse confocal imaging was performed of the control and cdh5-silenced lcr:eGFP×flk1:mCherry embryos.

It was found that lcr:eGFP⁺ red blood cells were accumulated in the blood vessels of cdh5-silenced embryos even after the heart begins to beat; demonstrating the absence of active circulation in cdh5-morphants, despite the initiation of heartbeat and formation of blood vessels.

To examine the function of the heart in the cdh5-silenced embryo, electrophysiology and echocardiography assessments were performed. The heart rate in the cdh5-MO embryos was comparable to the control, but stroke volume was near null in cdh5-MO embryos. Therefore, it was established that cardiac output (=stroke volume×heart rate) was impaired in cdh5-MO embryos.

The cdh5-MO embryos had pericardial edema in the cardiac cavities, which may be due to the back-flow of blood from the heart. The accumulation of fluid in the pericardial space results in a reduced ventricular filling and a subsequent hemodynamic compromise. To examine whether cardiac tamponade was a factor in the accumulation of fluid in the pericardial space, the cardiac cavity of cdh5-MO embryos were punctured, like in pericardiocentesis, and then pericardial fluid was aspirated to reduce the fluid-pressure buildup on the heart. However, the cardiac output deficiency of the cdh5-morphant heart could not be rescued.

Heartbeat was normal in cdh5-morphants, but their cardiac output was impaired due to structural defects in the heart, resulting in the accumulation of blood in the pericardial cavity. Since cdh5-MO embryos have normal hematopoiesis, it was hypothesized that the heartbeat-derived biomechanical forces influence HSC formation in the absence of active circulation.

Although cdh5-MO embryos have beating hearts and no active circulation, they have HSCs forming in the aortic endothelium of their blood vessels. When the AGM of control zebrafish embryos were zoomed in on, a distinct pulsation of the blood vessels was noticed. To distinguish the existence of pulsation in blood vessels independent of circulating blood cells and perhaps blood flow, the pulsation frequency of blood vessels with that of the circulating blood cells and movement due to the blood flow was compared. Specifically, the time-lapse confocal imaging of a double transgenic line with circulating lcr:eGFP⁺ red cells within flk1:mCherry⁺ blood vessels, as well as Fourier analysis of the signal from both blood vessels and from the circulating blood cells was performed. The frequency spectrum of blood vessels was found to have a distinct peak. Thus, the pulsation in blood vessels and the blood flow co-exist, but their existence and nature are independent of each other.

To investigate the temporal, spatial, and functional existence of pulsation in the AGM at 36 hpf, the light sheet microscopy of the blood vessels region in control zebrafish embryos followed by Fourier analysis was performed. The data further corroborate that the AGM has a distinct pulsation frequency at 36 hpf; which is the time and location for the endothelial-to-hematopoietic transition as seen with time-lapse confocal imaging of runx1:mCherry⁺ HSPCs emerging from flk1:eGFP⁺ endothelial cells. Together, the AGM region is found to be pulsating and the pulsation in the AGM is concurrent with the endothelial-to-hematopoietic transition.

Blood vessels are under constant mechanical loading from heartbeat-mediated blood pressure and flow, which cause circumferential wall stress and endothelial shear stress. While blood flow imposes shear stress on endothelial cells and induces vasodilation, heartbeat-mediated pulsation generates circumferential stretch and causes mechanical distension on both endothelial cells and smooth muscle cells.

To analyze if cdh5-MO embryos form HSCs through or are independent of blood flow- and shear-stress mediated NOS activation, HSPC expression was analyzed in control and cdh5-MO embryos treated with L-NAME, a NOS inhibitor. It was demonstrated that the inhibition of NOS attenuates HSPC formation in control embryos, but it does not impact HSPC formation in cdh5-MO embryos. Therefore, cdh5-MO embryos form HSCs independent of NOS activation.

Taken altogether, heartbeat-mediated pulsation stimulates the endothelial-to-HSC formation independent of circulation.

Stretch activates Piezo1 for HSC formation.

Since biomechanical forces stimulate cell shape and fate transitions, it was hypothesized that the pulsation-mediated periodical stretching of the hemogenic endothelium stimulates HSC formation.

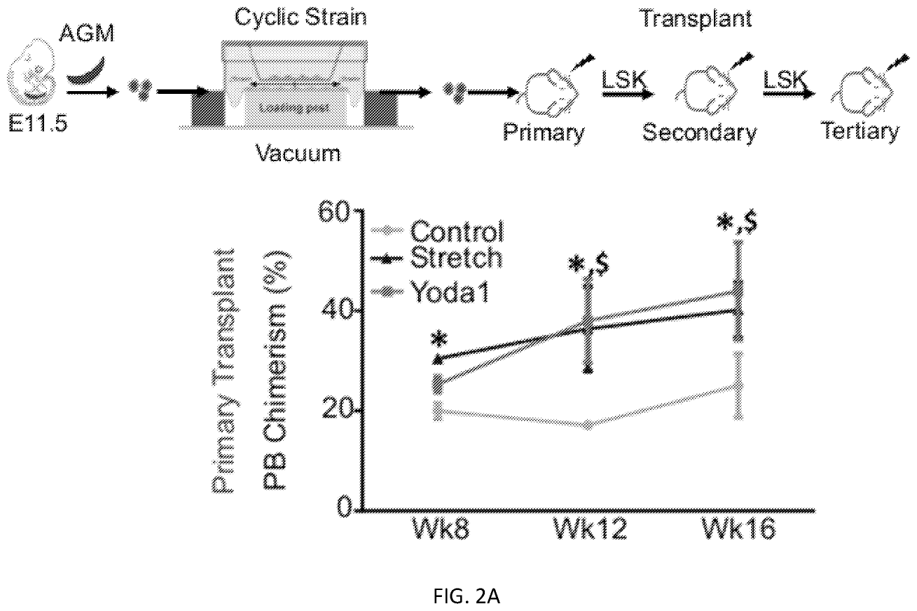

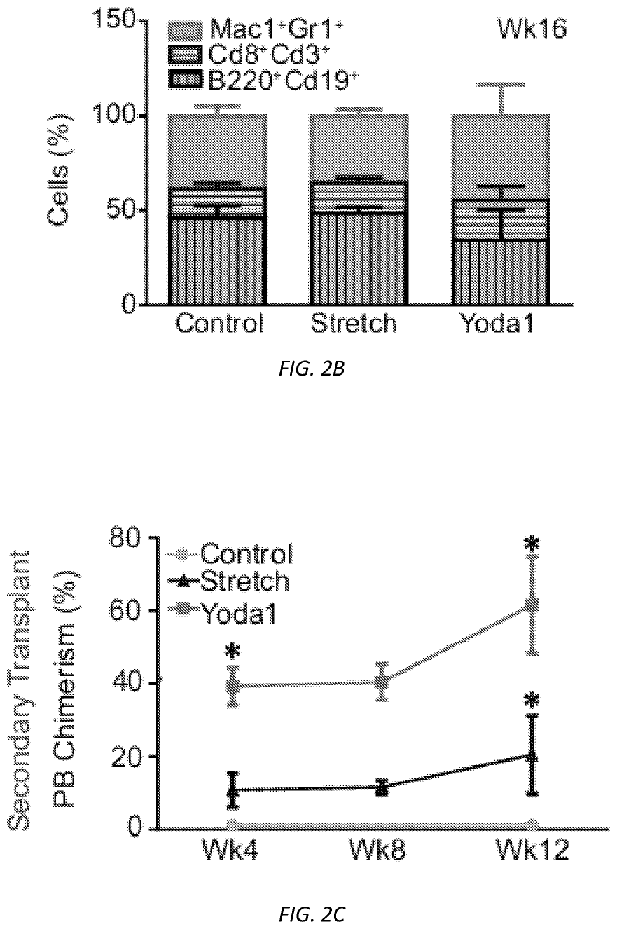

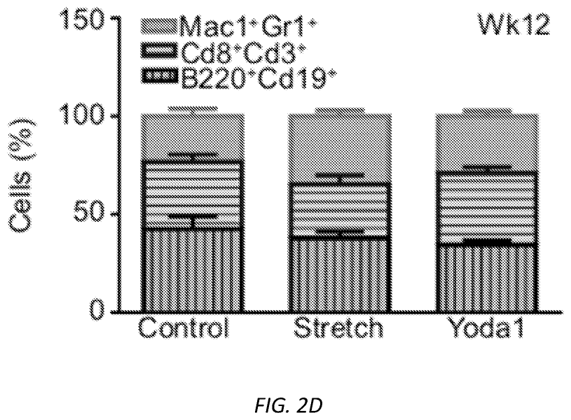

To test the function of pulsation in endothelial-to-HSC formation, a bioreactor was developed that could apply cyclic strain on AGM cells harvested from E11.5 mice embryos (FIG. 2A, top panel). Hematopoietic colony formation and flow analyses assays demonstrated that 10% cyclic strain potentiates the formation of multipotent hematopoietic progenitors, which is attenuated by GdCl₃-mediated pan-pharmacological inhibition of stretch-activated receptors (SAR). GdCl₃ also attenuated HSPC expression in zebrafish embryos to the level of sih-MO embryos.

The SAR family members have four sub-categories: K1-family members as well as Piezo, TRP, and DEG/ENaC channels. Tissue expression and computational analyses display Piezo1 and Trpv4 in endothelial and hematopoietic tissues, so their roles were tested in the endothelial-to-HSC transition.

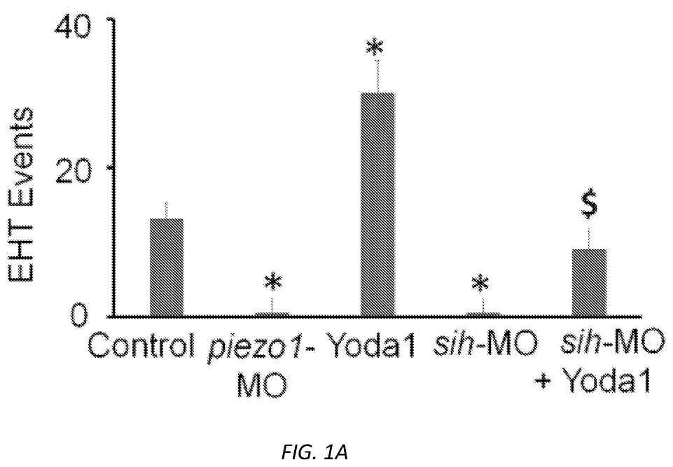

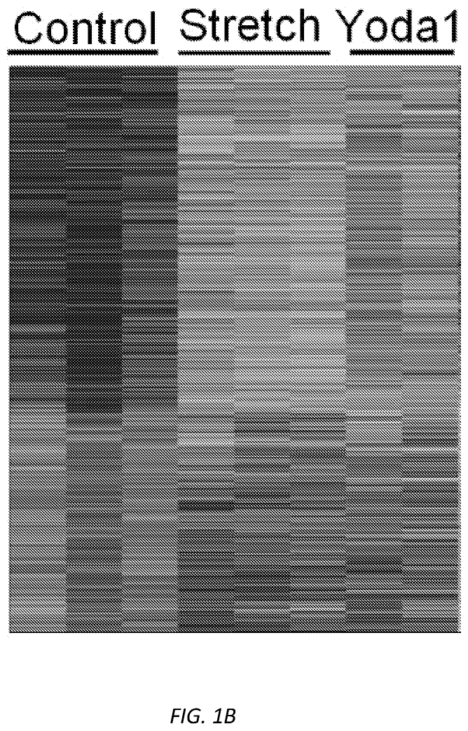

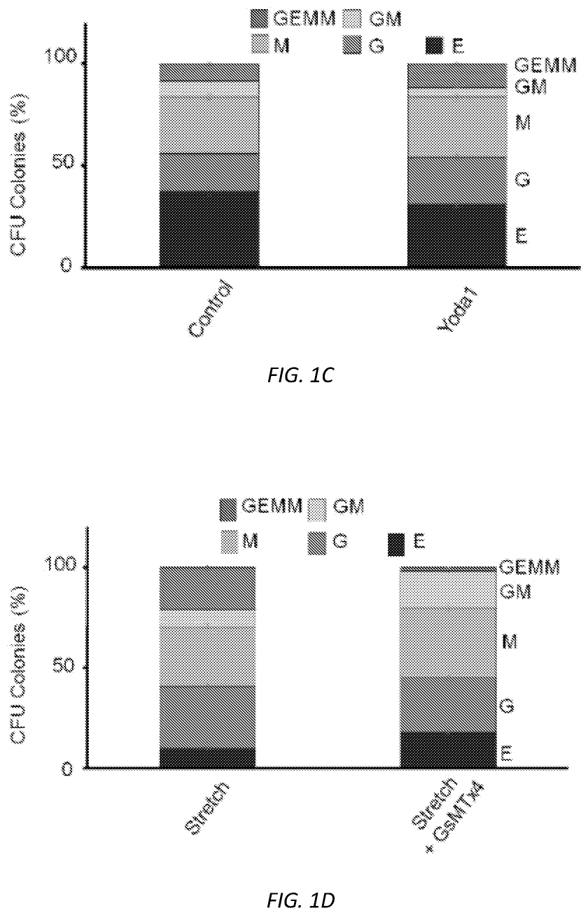

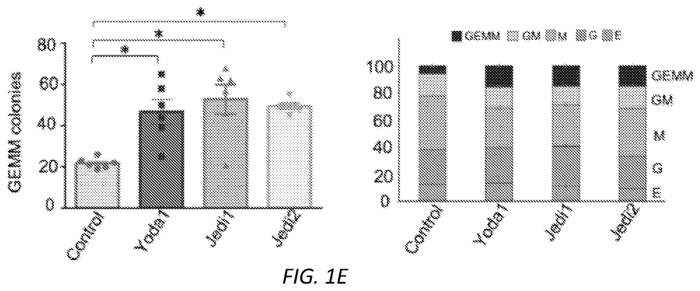

The loss-of-function analyses and pharmacological inhibition of trpv4 and piezo1 abolished HSPC marker expression and the endothelial-to-HSC transition (FIG. 1A). Conversely, pharmacological activation of trpv4 or piezo1 enhanced HSPC marker expression in control embryos, and rescued HSPC expression in sih-embryos. Upon temporal and spatial analyses, trpv4 was not detected in the AGM region of zebrafish embryos at 36 hpf, whereas Piezo1 co-localized with Cd31 (endothelial) and c-Kit (hematopoietic) in E11.5 AGM.

To consolidate the molecular mechanism underlying stretch-mediated HSC formation, whole transcriptome analyses of AGMs treated with either cyclic strain or a pharmacological activator of Piezo1 was performed. It was found that cyclic strain and Piezo1 activation produced similar gene signatures (FIG. 1B).

The pharmacological activation of Piezo1 further enhanced multipotent hematopoietic progenitor cell formation (FIG. 1C), whereas the pharmacological inhibition of Piezo1 attenuated the cyclic strain-mediated induction of HSPC formation (FIG. 1D). Together, cyclic strain-mediated biomechanical stretching activates Piezo1 to stimulate the endothelial-to-HSC transition.

Similar results were obtained with the Piezo1 agonists Yoda1, Jedi1, and Jedi2. Specifically, as shown in FIG. 1E, hematopoietic CFU assays on E11.5 AGM cells treated with 50 μM Jedi1, 50 μM Jedi2, or 25 μM Yoda1 demonstrated that Jedi1, Jedi2, or Yoda1-mediated Piezo1 activation enhances GEMM formation.

### Biomechanical Stretching or Piezo1 Activation Produces LT-HSCs.

To analyze if cyclic strain or Piezo1 activation produces long-term, self-renewing HSCs (LT-HSCs), serial transplantation assays were performed. The primary transplant of cyclic strain or Piezo1 activator treated AGMs displayed higher engraftment and normal multi-lineage reconstitution (FIG. 2A, FIG. 2B). Also, the bone marrow of primary recipients transplanted with cyclic strain or Piezo1 activator treated AGMs displayed two- to three-times higher amount of Lin⁻Sca1⁺c-Kit⁺Cd48⁻Cd150⁺ HSCs (i.e., LT-HSCs). The transplantation of primary recipient-derived sorted Lin⁻Sca1⁺c-Kit⁺ HSPCs into immunocompromised secondary recipients also resulted in higher engraftment and normal multi-lineage reconstitution (FIG. 2C, FIG. 2D). Therefore, it was predicted that both cyclic strain and/or Piezo1 activation produce higher amounts of normal LT-HSCs. To test this hypothesis, a limiting dilution assay was performed by transplanting graded amount of Lin⁻Sca1⁺c-Kit⁺ HSPCs into immunocompromised tertiary recipients. The tertiary transplant analyses demonstrated that cyclic-strain produced two- to three-times higher amount of LT-HSCs.

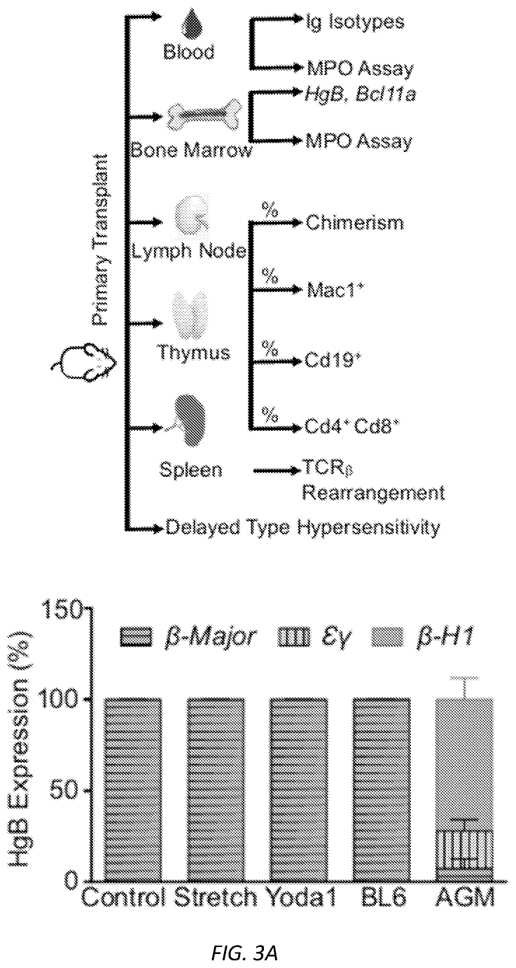

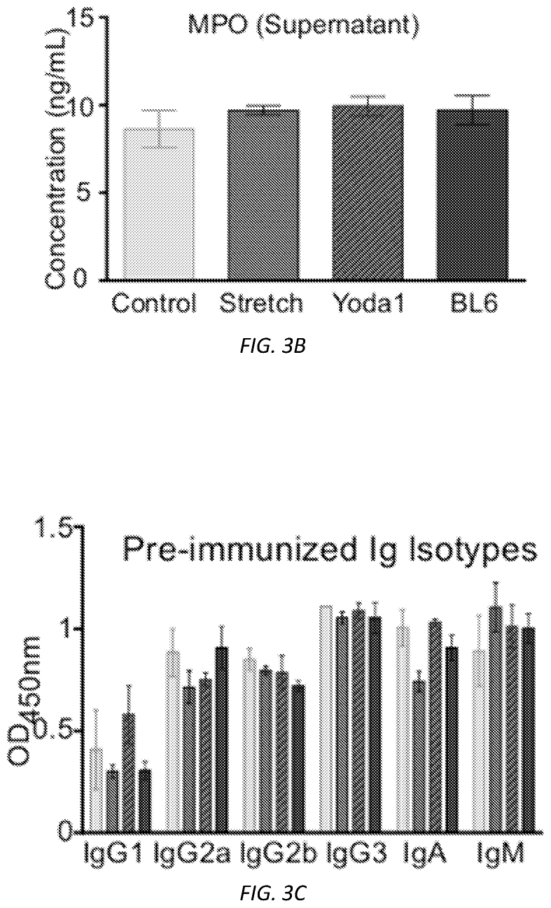

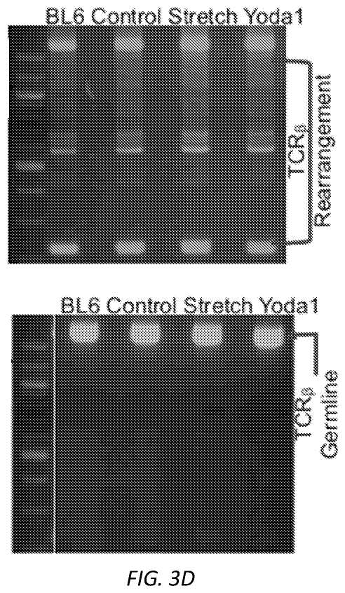

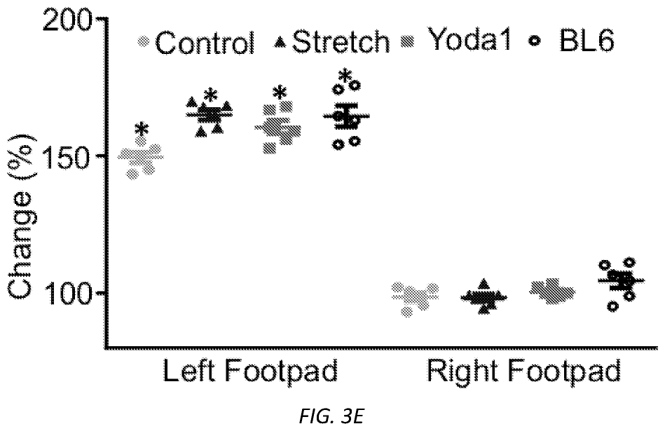

To investigate if AGM-HSCs (donor) engraft and reconstitute to adult normal blood, the molecular features and functional properties of reconstituted blood lineages were then analyzed in the primary recipients transplanted with control, cyclic strain or Piezo1 activator treated AGMs. The analysis of donor-derived erythroid cells in the bone marrow displayed Cd71⁺/Ter119⁺ expression, as well as enhanced expression of adult globin markers at the cost of embryonic globin in the presence of Bcl11a (FIG. 3A). Further analysis of donor-derived myeloid cells in the bone marrow and blood serum displayed sufficient amounts of Gr1⁺/Mac1⁺ myeloid cells, as well as their production of myeloperoxidase (MPO) (FIG. 3B). Next, analyses of donor-derived chimerism, Mac1⁺ myeloid cells, Cd19⁺ B-cells, as well as Cd4⁺/Cd8⁺ T-cells in the lymph node, thymus, and spleen demonstrated that donor HSC-derived progenitors circulated and colonized in the hematopoietic niches to reconstitute to adult blood lineage. Upon analyses of primary transplant-derived blood serum, it was also found that they produced the normal repertoire of pre-immunized immunoglobulins (Ig), such as IgG1, IgG2a, IgG2b, IgA, and IgM (FIG. 3C). The sorting of donor-derived Cd3⁺ T-cells from the spleen demonstrated T-cell receptor (3 (TCR (3) rearrangement, which was absent in donor-derived Mac1⁺ myeloid cells (negative control) from the spleen (FIG. 3D). To analyze the functional properties of T-cells in primary transplant, the delayed-type hypersensitivity assay demonstrated the successful recruitment of antigen-specific functional T-cells in footpad, by sensitizing primary transplant with sheep red blood cell injection (FIG. 3E). Thus, cyclic strain or Piezo1 activation of AGMs or hemogenic endothelial cells produced HSCs that engrafted in hematopoietic niches and reconstituted to functional, multi-lineage adult blood.

### Biomechanical Stretching and Piezo1 Activation Upregulate Dnmt3b for the Endothelial-to-HSC Transition.

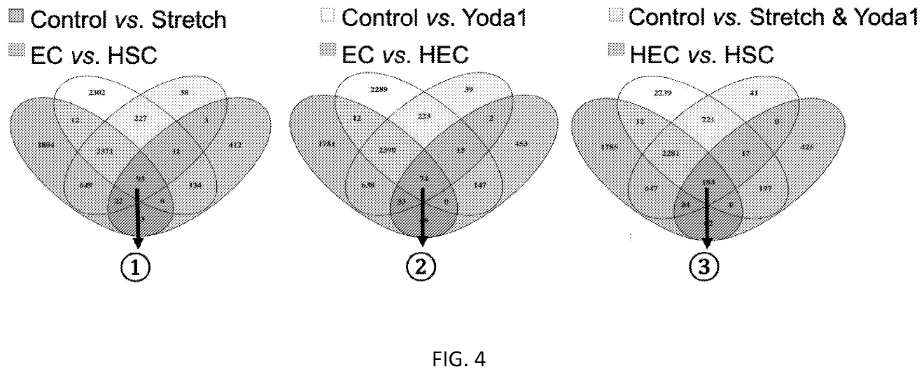

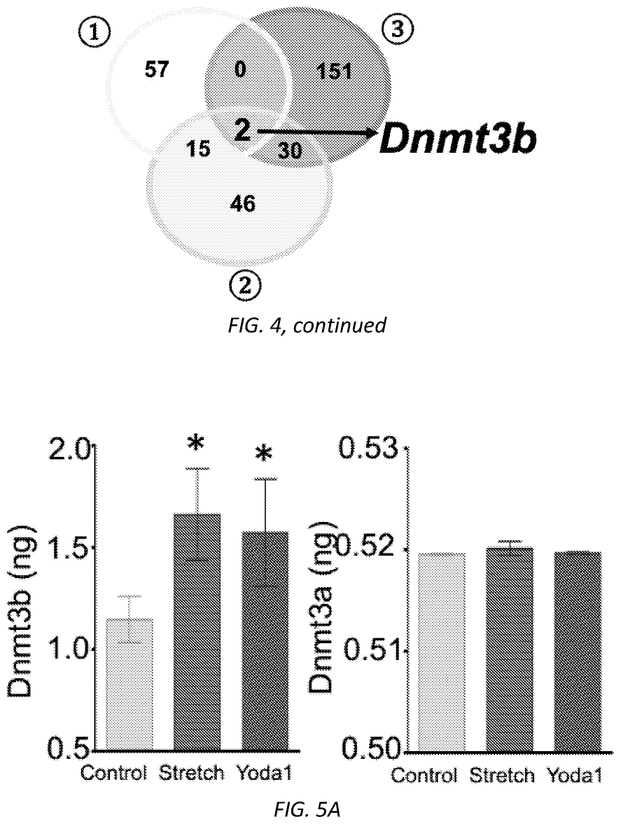

Since the AGM is a heterogeneous tissue, it was unclear how stretch-mediated Piezo1 activation would stimulate the aortic endothelial cell fate transition to HSCs. Differential gene expression signatures from E10.5 AGM-sorted endothelial cells, hemogenic endothelial cells, and HSCs were developed. Hierarchical clustering of gene signatures derived upon cyclic strain or Piezo1 activation of the AGM in the context of AGM-derived endothelial cells, hemogenic endothelial cells, and HSCs further provided the quantitative overview of overexpressed biological processes, molecular pathways, gene expression clusters, and their gene ontology (GO) terms. Venn diagram analyses of cyclic stretch and/or Piezo1 activation-mediated genes upregulated during the endothelial-to-HSC transition identified Dnmt3b as a potential candidate mechanism responsible for the silencing of endothelial machinery required for HSC formation (FIG. 4). In addition, Gimap6 was also identified as a potential candidate mechanism responsible for the silencing of endothelial machinery required for HSC formation.

To validate the bioinformatics and computational analyses, the temporal and spatial protein expression of Dnmt3b in E11.5 AGM was analyzed. The immunohistochemistry assay demonstrated that Dnmt3b co-localizes with Cd31⁺ endothelial and c-Kit⁺ hematopoietic cells. Thus, it was hypothesized that Dnmt3b could stimulate the endothelial-to-HSC transition.

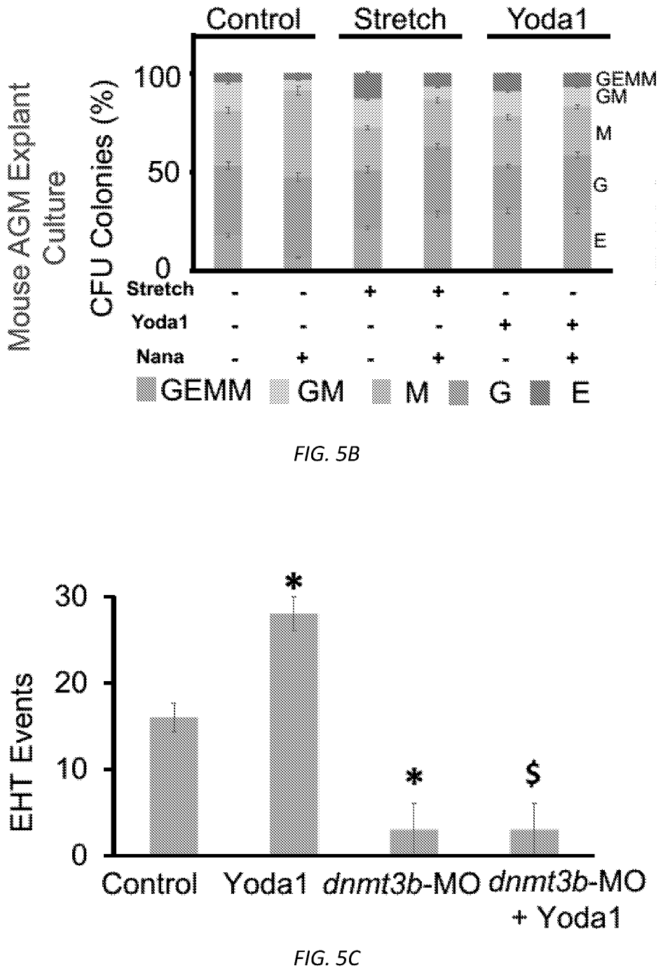

Although Dnmt3b and Dnmt3a are highly homologous and have distinct functions in HSC maintenance or differentiation, their potential roles in the endothelial-to-HSC in AGM were unknown. The gene signatures and tissue expression analyses excluded any involvement of Dnmt3a in HSC formation in the AGM. To distinguish the distinct or overlapping hemogenic role(s) of Dnmt3b and Dnmt3a, Dnmt3b and Dnmt3a protein levels were analyzed in nuclear fractions of cyclic strain- or Yoda1-treated AGM cells, which established that cyclic strain or Piezo1 activation stimulates Dnmt3b protein expression, and not Dnmt3a, in E11.5 AGM cells (FIG. 5A).

To analyze whether the pulsation of blood vessels, in the absence of blood flow, stimulated HSC formation via Dnmt3b activation, HSPC marker expression was measured in cdh5-MO embryos treated with Nanaomycin, a Dnmt3b inhibitor. The pharmacological inhibition of Dnmt3b attenuated HSPC marker expression in control and cdh5-MO embryos.

Next, the experiments of this example analyzed whether biomechanical stretching or Piezo1 activation stimulated the endothelial-to-hematopoietic transition via Dnmt3b activation. It was found that the inhibition of Dnmt3b attenuated the biomechanical stretching- or Piezo1 activation-mediated induction of multipotent hematopoietic progenitor cell formation (FIG. 5B), as well as the endothelial-to-hematopoietic transition (FIG. 5B). Although Nanaomycin treatment reverts hematopoietic cells into phenotypic endothelial cells, such endothelial cells were not functional. The whole mount in situ hybridization of HSPC markers, as well as time-lapse imaging of the endothelial-to-HSC transition in Nanaomycin-treated or dnmt3b-MO-injected zebrafish embryos concurrently treated with or without Yoda1 further validated that the inhibition or the loss of dnmt3b attenuated the Piezo1 activation-mediated increase in HSC formation (FIG. 5C). Together, pulsation-mediated Piezo1 activation enhanced Dnmt3b expression in the AGM to stimulate the endothelial-to-HSC transition.

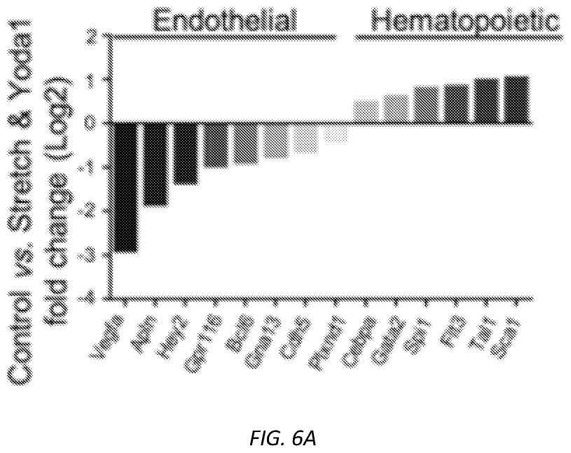

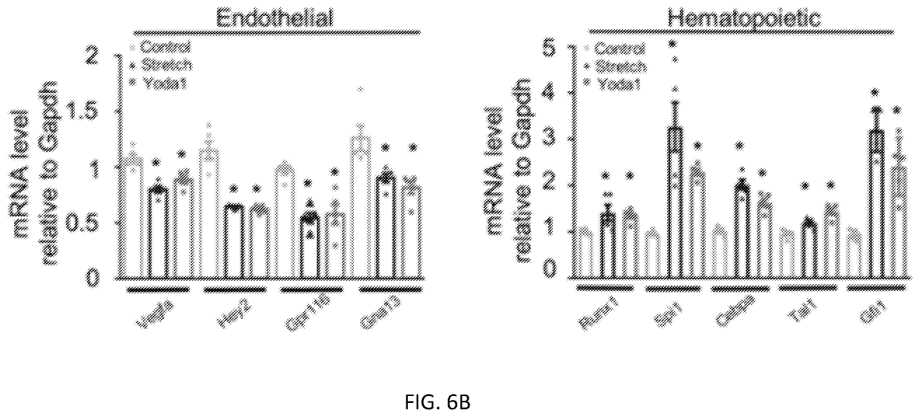

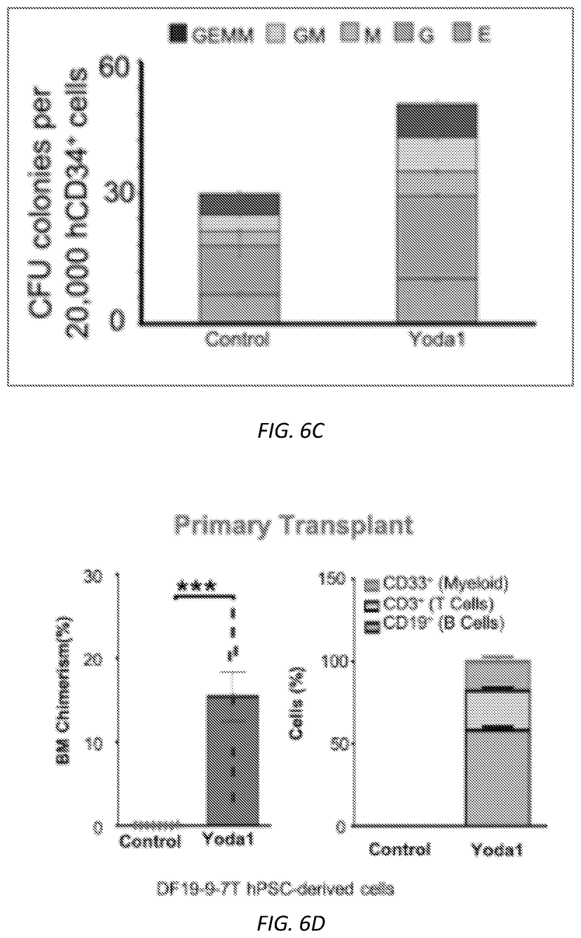

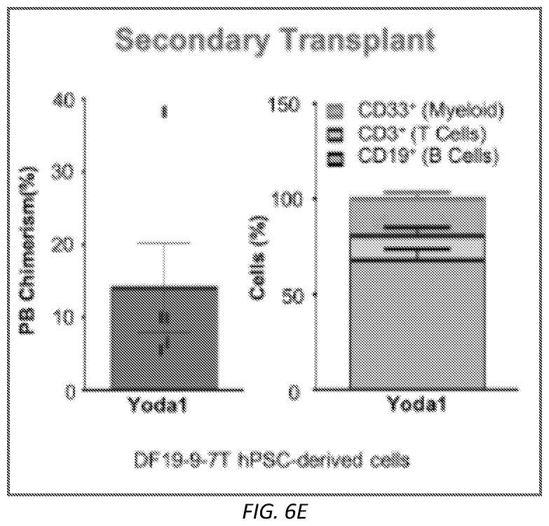

To determine the role of Dnmt3b in the EHT, we analyzed the endothelial and hematopoietic gene expression levels using whole transcriptome analyses. We found that 2D cyclic stretch- or Yoda1-treated AGM samples had diminished expression of endothelial genes (Vegfa, Apln, Hey2, Gpr116, Bcl6, Gna13, Cdh5, Plxnd1) and elevated expression of hematopoietic genes (Sca1, Tal1, Flt3, Spi1, Gata2, Cebpa) (FIG. 6A). To consolidate our findings, we further independently measured the transcript levels of endothelial and hematopoietic genes. We found that both cyclic strain- or Piezo1 activation-mediated Dnmt3b overexpression led to endothelial gene silencing (Vegfa, Hey2, Gpr116, Gnal3) and higher expression of hematopoietic genes (Runx1, Spi1, Cebpa, Tal1, Gfi1) (FIG. 6B) during the EHT. In summary, pulsation-mediated Piezo1 activation enhances Dnmt3b expression to repress endothelial genes, stimulating the endothelial-to-HSC transition. To analyze the conserved role of PIEZO1-mediated mechanosensitive mechanisms in human hematopoiesis, we employed directed differentiation of constitutive RUNX1c:tdTomato human induced pluripotent stem cells (iPSCs) to hemogenic endothelial cells, and treated such hemogenic endothelial cells with Yoda1. We found that Yoda1-mediated PIEZO1 activation stimulated the human endothelial-to-hematopoietic transition. We also found that Yoda1-mediated PIEZO1 activation enhanced DNMT3B expression but not DNMT3A expression, silenced endothelial genes (VEGFA, HEY2, GPR116, GNA13, CDH5, PLXND1), and induced the expression of hematopoietic genes (RUNX1, SPI1, CEBPA, TAL1, GATA2); which led to elevated multipotent hematopoietic progenitor formation and human hematopoiesis (FIG. 6C). Thus, pulsation-mediated PIEZO1 activation stimulates the endothelial-to-hematopoietic transition in zebrafish, murine, and human model systems.

Further, as shown in FIG. 6D, Yoda1-mediated pharmacological activation of Piezo1 enhances the formation of engraftable human CD34+ cells, and that pharmacological activation of Piezo1 stimulates the formation of human CD34+ hematopoietic cells that reconstitute multi-lineage blood. Further, pharmacological activation of Piezo1 enhances the formation of self-renewing LT-HSCs that reconstitute multi-lineage blood upon serial transplantation. See FIG. 6E.

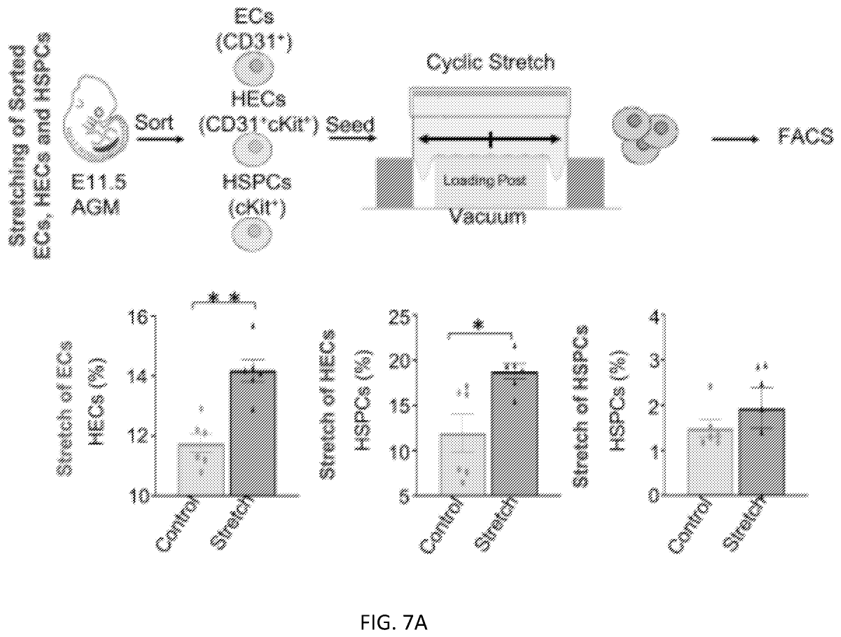

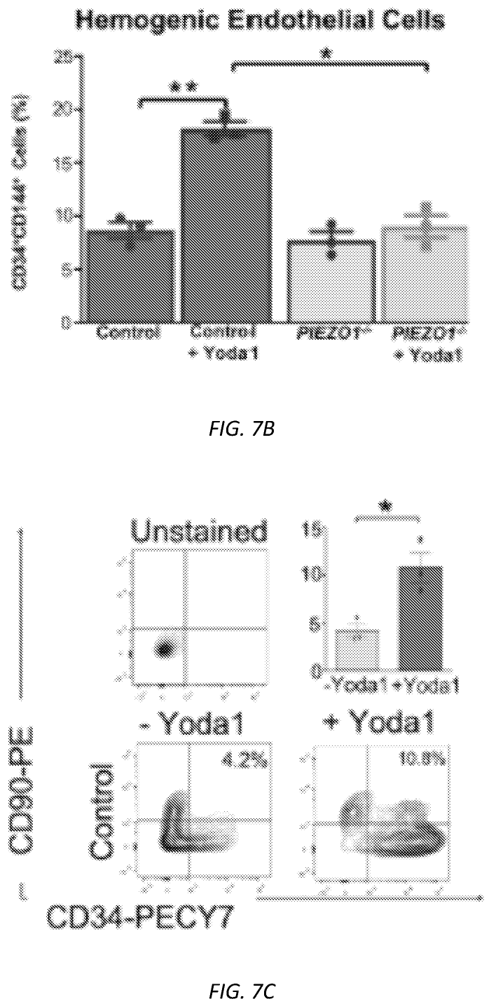

Cyclic stretch and Piezo1 agonists promote the EC to HSC transition, including the transition from ECs to HE cells, and the transition from HE cells to HSCs. FIG. 7A (top) illustrates a study where 10% cyclic stretch on mouse E11.5 AGM-sorted ECs (CD31⁺), HECs (CD31⁺cKit⁺) and HSPCs (cKit⁺) cells was followed by FACS analysis. As demonstrated in FIG. 7A (bottom), cyclic stretch promotes the EC to HE cell transition (left) as well as HEC to HSPC transition (center). The effect on HSPCs is shown on the right.

FIG. 7B shows FACS analyses of human-derived embryoid bodies (EBs) at day 8 of hematopoietic differentiation, showing that Yoda1-mediated PIEZO1 activation enhances the formation of hCD43^{neg}CD235^{neg}CD144⁺CD34⁺ HE cells in control but not in PIEZO1^{-/-} PSCs. Yoda1-mediated Piezo1 activation enhances formation of HSCs from hPSC. FIG. 7C. FIG. 7C shows a 2-Fold improvement in the number of HSCs produced from hPSCs using Yoda1-mediated Piezo1 activation.

Production of HSCs from HE Cells Generated from Human iPSCs

Embryoid body and hemogenic endothelium differentiation was performed as described in (Sugimura et al. 2017; Ditadi et al. 2015). Briefly, hiPSC colonies were dissociated with 0.05% trypsin for 5 min at 37° C., washed with PBS+2% FBS, and resuspended in StemPro-34 (Invitrogen, 10639-011) supplemented with L-glutamine (2 mM), penicillin/streptomycin (10 ng/ml), ascorbic acid (1 mM), human holo-Transferrin (150 m/ml, Sigma T0665), monothioglycerol (MTG, 0.4 mM), BMP4 (10 ng/ml), and Y-27632 (10 μM). Five million cells were seeded into 10 cm dishes (Ezsphere, Asahi Glass) for the spheroid formation. On Day 1, bFGF (5 ng/ml) and BMP4 (10 ng/ml) was added to the medium. On Day 2, the media was changed with the StemPro-34 supplemented with SB431542 (6 μM), CHIR99021 (3 μM), bFGF (5 ng/ml), and BMP4 (10 ng/ml). On Day 3, the medium was replaced with StemPro-34 supplemented with VEGF (15 ng/ml) and bFGF (10 ng/ml). On day 6, the medium was changed to StemPro-34 supplemented with bFGF (5 ng/ml), VEGF (15 ng/ml), interleukin (IL)-6 (10 ng/ml), IGF-1 (25 ng/ml), IL-11 (5 ng/ml), SCF (50 ng/ml) and EPO (2IU). The cells were maintained in a 5% CO₂, 5% O₂ and 95% humidity incubator. All cytokines were purchased from Peprotech.

To isolate the CD34⁺ cells, the embryoid bodies (from day 8) were dissociated by 0.05% trypsin, filtered through a 70 μm strainer, CD34⁺ cells were isolated by CD34 magnetic bead staining, and subsequently passaged through the LS columns (Miltenyi). A sample from every batch was tested by FACS to validate its purity with the panel. The following antibodies were employed: CD34-PEcy7 (Clone 581; Biolegend), FLK1-PE (CLONE #89106; BD), and 4′,6-diamidino-2-phenylindole (DAPI).

Isolated CD34⁺ cells were resuspended in StemPro-34 medium containing Y-27632 (10 μM), TPO (30 ng/ml), IL-3 (10 ng/ml), SCF (50 ng/ml), IL-6 (10 ng/ml), IL-11 (5 ng/ml), IGF-1 (25 ng/ml), VEGF (5 ng/ml), bFGF (5 ng/ml), BMP4 (10 ng/ml), and FLT3 (10 ng/ml) (Ferrel et al 2015). Cells were seeded at a density of 50,000 cells per well onto thin-layer Matrigel-coated 24-well plates. One day after seeding, Yoda1 (between 6.25 and 100 μM) was added to the cultures. After 7 days, the floating cells were collected and FACS analysis performed. For FACS analysis, cells were stained with CD34-PEcy7 (Clone 581; Biolegend) and CD45-APC (clone 2D1; Biolegend). All the cytokines were purchased from Peprotech.

### Conclusions

The development, expansion, and maintenance of long-term HSCs have been a holy grail in stem cell biology and hematopoiesis. Based on time-lapse confocal, light sheet, and Fourier Transform analyses in zebrafish, not only was a scalable bioreactor simulating pulsation in blood vessels established, but also Piezo1 activation was identified as a pharmacological target to transform endothelial cells into LT-HSCs. This study provides a novel transgene-free approach to developing LT-HSCs that can engraft, self-renew, and reconstitute to multi-lineage, functional, adult blood upon serial transplantation.

Heartbeat-mediated pulsation generated circumferential stretch and caused mechanical distension on both endothelial cells and smooth muscle cells. However, Piezo1 was co-expressed between endothelial and hematopoietic cells in E11.5 AGM, but not in vascular smooth muscle cells of blood vessels, which suggested that the hemogenic role of biomechanical stretching and Piezo1 activation is intrinsic to AGM-endothelial cells.

Biomechanical stretching of blood vessels could activate Piezo1, Trpv4, K1-family members, as well as DEG/ENaC channels. Both Piezo1 and Trpv4 activation stimulated the endothelial-to-hematopoietic transition. However, only Piezo1 inhibition attenuated the stretch-mediated hemogenic effect, which suggested that Piezo1 and Trpv4 may have partially redundant roles.

Dmnt3b activation silenced the endothelial machinery to endow HSCs with self-renewal and multi-lineage reconstitution capacity. Although the inhibition of Dnmt3b reverts hematopoietic cells to phenotypic endothelial cells, these cells lacked functional endothelial properties. This suggested that the temporal and spatial role of Dnmt3b in the endothelial-to-hematopoietic transition was non-reversible. Biomechanical stretching or Piezo1 activation enhanced temporal and spatial expression of Dnmt3b without impacting Dnmt3a expression. The data demonstrated a distinction between the hemogenic role of Dnmt3b and the leukemic role of Dnmt3a during HSC development and differentiation.

The findings disclosed herein demonstrate how biomechanical forces stimulate cell fate transition and endow self-renewing capacity to stem cells by invoking epigenetic machinery. This study also provides a platform to derive LT-HSCs from pluripotent stem cells (PSC) or donor cell-derived endothelial or hemogenic endothelial cells. While a goal is to develop universally compatible HSCs, the bio-inspired bioreactor disclosed herein is a stepping stone when universally compatible, transgene-free source cells become available to treat patients with benign and malignant blood, metabolic, immune, and bone marrow diseases.

### Materials and Methods

All procedures were approved by the Animal Care and Use Committees of Brigham and Women's Hospital and Boston Children's Hospital.

Mice were purchased Cd45.2 (C57BL6/J) and Cd45.1 (SJL) from The Jackson Laboratory and zebrafish morpholinos from GeneTools. Microangiography was performed by injecting fluorescent-labeled dextran dye in the atrium of zebrafish heart and its passage was recorded using live imaging. Immunostaining of zebrafish heart and mouse AGM were analyzed using an inverted fluorescent microscope. Cardiac tamponade, heart rate, and pulse frequency were analyzed in zebrafish embryos using bright field imaging or time-lapse confocal microscopy. The movement of red blood cells in blood vessels was analyzed as well as the endothelial-to-HSC transition in zebrafish transgenic embryos using time-lapse confocal imaging.

Pulsating blood vessels like conditions were stimulated in vitro using Flexcell FX-4000 machine. To analyze roles of pharmacological targets in regulating the endothelial-to-HSC transition, mouse embryo-derived AGM or whole mouse embryo were exposed ex vivo with biomechanical stretching, chemicals, or drugs. Next, hematopoietic colony formation assays were performed by incubating mouse AGM-derived cells in StemCell M3434 media for seven days. Serial transplantation of AGM-derived HSCs in lethally irradiated SJL mice were performed. The stem cell frequency upon biomechanical stretching was analyzed using a limiting dilution assay. To characterize properties of AGM-HSC-derived blood cells in primary transplants, percentage chimerism and reconstitution was measured using FACS, globin transcripts were analyzed using quantitative reverse transcriptase-PCR, myeloperoxidase amount was measured using PicoKine ELISA kit, TCR-β rearrangement was analyzed using PCR for TCR-β locus, pre-immune IG detection was analyzed using Thermo-Fisher Mouse Ig Isotyping kit, and delayed-type hypersensitivity was analyzed by injecting sheep RBC (Rockland Immunochemicals) in the footpad of pre-sensitized mice.

RNA-sequencing analyses were performed to measure gene expression patterns in mouse AGM treated with cyclic strain or pharmacological modulators. Using computational algorithms, hierarchical clustering was performed of differentially expressed genes as well as measured their overrepresented biological processes and pathways. Gene expression clusters of differentially expressed genes were analyzed and their mean expression level across cell populations compared. Next, Venn comparison of up- and down-genes was constructed to analyze candidate(s) important for cyclic strain- or pharmacological modulator(s)-mediated the endothelial-to-HSC transition. Furthermore, Dnmt3b and Dnmt3a protein expressions were analyzed in nuclear fractions of mouse AGM cells using EqiQuick assay kits. Data are presented as mean±s.d. unless otherwise noted. Statistical analyses were performed by paired or un-paired Student's t-tests. Significance was set at P<0.05.

Animals

Experiments used wild-type AB, Casper, and transgenic zebrafish lines lcr:eGFP, flk1:mCherry, flk1:eGFP, cd41:eGFP. Embryos were used up to 4 days pf. Experiments used Cd45.2 (C57BL6/J) and Cd45.1 (SJL) mice from The Jackson Laboratory.

Morpholinos

Morpholino antisense oligos were obtained (Gene Tools; sequences below) and injected into one-cell stage casper zebrafish embryos. Injected and uninjected embryos were incubated in E3 media at 28° C. until fixation.

| Target | Sequence (5′-3′) | SEQ ID NO: |
|---|---|---|
| cdh5-MO | TACAAGACCGTCTACCTTTCCAATC | 1 |
| sih-MO | CATGTTTGCTCTGATCTGACACGCA | 2 |
| piezo1-MO | CAAAGTTCAGTTCAGCTCACCTCAT | 3 |
| dnmt3bb.1-MO1 | TTATTTCTTCCTTCCTCATCCTGTC | 4 |
| dnmt3bb.1-MO2 | CTCTCATCTGAAAGAATAGCAGAGT | 5 |

Chemical Treatment of Embryos

Zebrafish embryos were treated with the following chemical modulators in E3 fish media: 100 uM L-NAME (Fisher Scientific), 50 uM Digitoxigenin (Sigma), 25-50 uM Yoda1 (Cayman Chemical), 1 uM Nanaomycin (Nana; Fisher Scientific), 100 uM Gadolinium chloride (GdCl₃; Sigma), 5-10 uM 4α-phorbol 12, 13-didecanaote (4Apdd; Sigma), or GSK205 (10 uM).

Microangiography

Fluorescent dye-labeled dextran beads were injected into the atrium of the control and cdh5-MO embryos, and captured real-time brightfield videos using a Nikkon SMZ1500 stereo microscope.

Heart Rate and Cardiac Output

Images of live zebrafish hearts were acquired on an Axioplan (Zeiss) upright microscope with a 5× objective lens using integrated incandescent illumination and a FastCam-PCI high-speed digital camera (Photron) with a 512×480 pixel grayscale image sensor. Images were obtained at 250 frames per second, with 1088 frames (′8 cardiac cycles) being acquired per condition. A custom software was used (implemented in MATLAB) to determine heart rate from sequential image files. Ventricular long and short axis were measured in both diastole and systole manually for each video using ImageJ and used to estimate chamber volume using standard geometric assumptions. A cardiac output was measured as diastolic minus systolic ventricular volume multiplied by heart rate (Shin et al., 2010), for at least ten embryos per morpholino dose.

Periodicity Analyses

Zebrafish Casper embryos were embedded in 0.8% low-melting-point agarose with tricaine (Sigma) and mounted in a petri dish. Next, a Nikon SMZ1500 stereomicroscope equipped with NIS Elements (Nikon) software was used to capture real-time brightfield videos of pulsating blood vessels in AGM region. The videos were used to quantify the pulse frequency in the blood vessels.

Brightfield Live Imaging

To perform brightfield live imaging, zebrafish Casper embryos were embedded in 0.8% low-melting-point agarose with tricaine (Sigma) and mounted in a petri dish. A Nikon SMZ1500 stereo microscope equipped with NIS Elements (Nikon) software was used to capture real-time brightfield videos and still images.

Confocal Microscopy

cd41:eGFP were crossed with flk1:mCherry zebrafish and flk1:mCherry with lcr:eGFP zebrafish and injected morpholino in their transgenic embryos. Transgenic embryos were mounted in low-melting-point agarose and a spinning-disk confocal microscope was used to perform time-lapse confocal imaging of cd41:eGFP⁺ HSCs emerging from flk1⁺ endothelium from 30 to 42 hpf. The relative movement of lcr:eGFP⁺ red blood cells was analyzed in the context of flk1:mCherry⁺ endothelium. We performed image analysis using Imaris (Bitplane) software.

Whole-Mount In Situ Hybridizations

Whole mount in situ hybridizations was performed as previously described.

Cardiac Tamponade

A microinjection needle was used to puncture the pericardial sac and release the fluid built up around the heart of cdh5-MO-injected zebrafish embryos at 48 hpf.

Immunostaining

E10.5 chimeric mouse embryos were harvested, embedded in a paraffin block, transverse sections performed, and immunostained with primary antibodies Piezo1 (rabbit anti-mouse IgG; Abcam), Cd31 (donkey anti-mouse IgG; R&D Systems), c-Kit (rabbit anti-mouse IgG; R&D Systems), or Dnmt3b (donkey anti-mouse IgG; Abcam). and 4,6 diamidino-2-phenylindole (DAPI) antibodies as well as secondary antibodies Alexa Fluor 488 (donkey anti-rabbit IgG; Fisher Scientific) and Alexa Fluor 647 (donkey anti-goat IgG; Abcam) to detect their expression in the E10.5 AGM region.

Expression of flk1 (GFP), mf2 (mCherry), and DAPI (violet) were measured in hearts isolated from control and cdh5-MO silenced zebrafish embryos.

AGM Explants

E11.5 AGM were harvested from C57BL6/J Cd45.2 mouse embryos, and a single cell suspension of a three-embryo equivalent of cells was seeded on each well of a BioFlex six-well culture plate (FlexCell). We cultured cells overnight with the application of cyclic strain (Flexcell® FX4000™ Tension System) and/or treatment with chemical modulators (2-100 μM Yoda1, 1 μM Nanaomycin, 100 μM Gdcl₃, 1 uM GsMT×4, 5-20 μM 4αPDD, 10 uM GSK205). Next, harvested cells were used to perform transplant, fluorescence-activated cell sorting (FACS) analysis, and colony-forming unit (CFU) assays.

Ex Vivo Incubation of the Embryos with Drugs

E11.5 mouse embryos were harvested from the uterus of the time-mated pregnant female, into sterile glass vials containing FBS, 1 mM glucose, 1% Penicillin-Streptomycin, and/or the selected chemical modulator (2-100 μM Yoda1, 1 μM Nanaomycin, 5-20 μM 4αPDD, or 10 uM GSK205). We placed glass vials in the ex vivo incubator (BTC Engineering, Cambridge, UK) consisting of a roller apparatus (rotating ˜30 rpm), constant gas supply (21% 02, 5% CO₂, balance N2) and constant temperature at 37° C. After 24 hours, AGM were harvested to analyze the formation of hematopoietic cells by FACS and CFU assays.

Transplants

For primary transplantation, three-embryo equivalents of untreated or treated (cyclic strain or 25 μM Yoda1) AGMs plus splenic helper cells (˜500,000 per mouse) were injected into lethally irradiated (split dose 10.5 cGy) Cd45.1 (SJL) mice by retro-orbital injection. For secondary and tertiary transplants, the bone marrow was isolated (legs, arm, pelvic bone, spine, sternum) from the transplanted mice. The bone marrow was loaded on a Ficoll gradient (Histopaque®-1083, Sigma-Aldrich), and the cells from the buffy coat incubated with biotin-conjugated lineage antibodies and streptavidin microbeads (Miltenyi Biotec). Next, the lineage negative (Lin⁻) cells were separated with MACS LS Columns (Miltenyi Biotec), and the donor Cd45.2 Lin⁻Sca1+c-Kit⁺ (LSK) cells sorted with a MoFlo Beckman Coulter sorter. Subsequently, the sorted Cd45.2 LSK cells were mixed with Cd45.1 splenic helper cells (˜500,000 per mouse) and transplanted into Cd45.1 irradiated (split dose 10.5 cGy) SJL mice by retro-orbital injection.

Survival recipients were counted as a response for the limiting dilution assay: confidence interval of 1/(stem-cell frequency) was calculated by ELDA, according to Poisson distribution.

CFU and FACS assay

For CFU assays, cells from AGM explant or ex vivo were plated in MethoCult GF M3434 media (StemCell Technologies). Seven days after seeding, we analyzed their capacity to make granulocyte, erythroid, macrophage, megakaryocyte (GEMM), granulocyte macrophage (GM), granulocyte (G), macrophage (M), and erythroid (E) colonies.

AGM cells from explants and ex vivo were stained with Sca1-Pacific-Blue (E13-161.7, Biolegend) and Flk1-APC-Cy7 (Avas 12α1, BD). Blood from transplanted mice was stained with the following antibody cocktail: Cd45.2-Pacific-Blue (104, Biolegend), Cd45.1-FITC (A20, Biolegend), Cd3-PE (145-2C11, Biolegend), Cd8-PE (53-6.7, Biolegend), Mac1-APC (M1/70, Biolegend), Gr1-APC (108412, Biolegend), Cd19-APC-CY7(6D5, Biolegend), B220-APC-CY7 (RA3-6B2, Biolegend).

Cells from the bone marrow, spleen, thymus, and lymph node from mice transplanted with E11.5 AGM cells were stained with the following antibody panels: Bone marrow LT-HSC: Cd45.2-FITC (104, Biolegend), Ter119-Biotin (TER-119 BD), Gr1-Biotin (RB6-8C5, BD), Cd5-Biotin (53-7.3, BD), Cd8a-Biotin (53-6.7, BD), B220-Biotin (RA3-6B2, BD), Streptavidin-Pacific Blue (eBioscience), Sca1-PE-CY7 (D7, eBioscience), cKit-APC (2B8, eBioscience), Cd48-APC-CY7 (HM48-1, BD), Cd150-PE-CY5 (TC15-12F12.2, Biolegend). Erythroid development RI-RV in bone marrow: Cd45.2-Pacific-Blue (104, Biolegend), Cd45.1-FITC (A20, Biolegend), Ter119-APC (TER-119, Biolegend), Cd71-PE (R17217, eBioscience). Bone marrow granulocytes: Cd45.2-Pacific-Blue (104, Biolegend), Cd45.1-FITC (A20, Biolegend), Gr1-PE, (RB6-8C5, BD); Mac1-APC (M1/70, Biolegend). Spleen, thymus and lymph nodes T Cells: Cd45.2-Pacific-Blue (104, Biolegend), Cd45.1-FITC (A20, Biolegend), Cd8-PE (53-6.7, Biolegend), Cd4-APC (RM4-5, eBioscience). Spleen, thymus and lymph node myeloid and B cells: Cd45.2-Pacific-Blue (104, Biolegend), Cd45.1-FITC (A20, Biolegend), Cd19-APC-CY7(6D5, Biolegend), Mac1-APC (M1/70, Biolegend). We performed all FACS analyses on a BD Fortessa cytometer. We performed hematopoietic organ analysis after 16 weeks of transplant.

Quantitative Reverse Transcriptase-Polymerase Chain Reaction Analysis (qRT-PCR)

FACS was used to sort erythroid precursors (Cd45.2⁺, Ter119⁺, Cd71⁺) from the unlysed bone marrow isolated from AGM-transplanted mice. Total RNA was isolated using the RNAeasy Minikit (QIAGEN) and cDNA synthesis performed using Superscript III (Invitrogen). Quantitative real-time PCR was performed using SYBR Green (QuantaBio) on an MX3000P machine with the indicated primers (Sankaran et al., 2009). We normalized the expression to that of glyceraldehyde-3-phosphate-dehydrogenase (Gapdh) (Ochida et al., 2010).

Myeloperoxidase (MPO) Expression

Neutrophils (Cd45.2⁺, Gr1⁺, Mac1⁺) were FACS sorted from the isolated bone marrow of 16 week-primary transplanted mice and cultured in IMDM with 10% FBS overnight (500,000 cells/mL) in 24-well plates. Supernatant was collected and the MPO concentration measured using the Mouse MPO/Myeloperoxidase PicoKine™ ELISA Kit (Boster). The MPO concentration was also measured in blood serum.

PCR Assay for TCR-β Rearrangement

T cells (Cd45.2⁺, Cd3⁺) and myeloid cells (Cd45.2⁺, Mac1⁺) were FACS sorted from the splenocytes of 16 week-primary transplanted mice. Next, genomic DNA was extracted, and PCR performed for DH (β2.1-JH (32.7 rearrangements within TCR-β locus. Our samples were denatured (94° C., 1 min), annealed (63° C., 2 mins) and extended (72° C., 2 mins) for 35 cycles. Primer sequences are as follows:

| Target | Sequence | SED ID NO: |
|---|---|---|
| 5′ of DH β2.1 | GTAGGCACCTGTGGGGAAGAAACT | 6 |
| 3′ of JH β2.7 | TGAGAGCTGTCTCCTACTATCGATT | 7 |

### See Lu et al., 2017.

Pre-Immune Ig Detection

Blood serum was isolated from 16 week-primary transplanted mice and pre-immune Ig isotypes were quantified by a mouse Ig isotyping kit (Thermo Fisher).

Delayed Type Hypersensitivity

Transplant mice were sensitized with sheep red blood cells (sRBC, 10⁹ cells/mL, 50 μL per site, Rockland Immunochemicals) through subcutaneous (lower back) and intradermal injections (right footpad). After six days of sensitization, pre-sensitized mice were challenged with 2×10⁹ sRBC/mL in the left footpad and an equal volume of PBS in the right footpad (as a control). After 48 hours of the challenge, the footpad thickness was measured with a micro-caliper. We normalized percent change at day 6 with the pre-challenged thickness of each footpad.

DNA Methyltransferase Expression

Nuclear extracts from AGM explants were harvested using an EpiQuik Nuclear Extraction Kit (Epigentek Group Inc.). Dnmt3b and Dnmt3a protein levels were analyzed using a colorimetric EpiQuik Assay Kit (Epigentek Group Inc.), according to the manufacturer's instructions. Concentration of Dnmt3b and Dnmt3a is relative to 1 μg of nuclear extract proteins.

RNAseq and Computational Analyses

Total RNA from E11.5 mouse AGM explant cultures was isolated (control, stretch, Yoda1 and 4αPDD conditions) with the RNAeasy MiniKit (QIAGEN). Our cDNA libraries were generated by BGI Americas Corporation and sequenced with a HiSeq4000 device (Illumina) at eight samples per lane. We mapped our sequenced read fragments to the mouse reference genome GRCm38 (ENSEMBL release 69) using the Genomic Short-Read Nucleotide Alignment program (version 2012-07-20). DESeq2 and DEXSeq were used to test for differential expression (FDR=0.1) and differential exon use, respectively. Gene expression clusters of differentially expressed genes were analyzed and their mean expression level across cell populations compared. Next, Venn comparison was performed of up- and down-genes to analyze candidate(s) important for cyclic strain- or pharmacological modulator(s)-mediating the endothelial-to-HSC transition. Specifically, we performed hierarchical clustering with bootstrap analyses using the gplots package (Warners et al., 2017) in R (R Development Core Team, 2012). For GO analysis, we tested for over-representation of our differentially expressed genes on GO categories or pathways using Fisher's exact test and corrected for multiple testing using the Bonferroni method. We performed GO term enrichment analyses as previously described using a P value of 0.001 as minimum for statistically significant enrichment.

### Statistical Analyses

Data are presented as a mean±standard error of the mean (Mean±SEM) unless otherwise noted. Statistical analyses were performed by paired or un-paired Student's i-tests. Significance was set at P<0.05.

What is claimed is:

1. A method of preparing a population of hematopoietic stem cells (HSCs) comprising Long Term (LT)-HSCs, the method comprising:
providing a population comprising endothelial and/or hemogenic endothelial (HE) cells,
decreasing expression or activity of two or more endothelial genes selected from vegfa, hey2, grp116, gna13, sox17, cdh5, plxnd1, bcl6, and apln in the endothelial and/or HE cells;
increasing expression or activity of two or more hematopoietic genes selected from runx1, spi1, cebpa, tal1, gfi1, gata2 and mllt3 in the endothelial and/or HE cells, so as to stimulate formation of the HSCs including LT-HSCs.

2. The method of claim 1, comprising decreasing expression or activity of three or more endothelial genes selected from vegfa, hey2, grp116, gna13, sox17, cdh5, plxnd1, bcl6, and apln in the endothelial and/or HE cells; and increasing expression or activity of three or more hematopoietic genes selected from runx1, spi1, cebpa, tal1, gfi1, gata2, and mllt3 in the endothelial and/or HE cells, so as to stimulate formation and optionally expansion of the HSCs.

3. The method of claim 1, comprising decreasing expression or activity of vegfa, hey2, grp116, gna13, cdh5, and plxnd1 in the endothelial and/or HE cells; and increasing expression or activity of runx1, spi1, cebpa, tal1, and gata2 in the endothelial and/or HE cells, so as to stimulate formation and optionally expansion of the HSCs.

4. The method of any one of claims 1 to 3, wherein the increasing of activity or expression of the hematopoietic genes comprises one or more of: introducing an encoding mRNA or an mRNA derivative, introducing an encoding transgene or episome, and introducing a genetic modification of expression elements or introducing a gain-of-function mutation to the hematopoietic gene.

5. The method of any one of claims 1 to 4, wherein the decreasing of expression or activity of endothelial genes comprises one or more of: introducing a full or partial gene deletion, RNA silencing, antisense oligonucleotide inhibition, pharmacological inhibition, and introducing a genetic modification of expression elements or introducing a loss-of-function mutation to the endothelial gene.

6. The method of any one of claims 1 to 3, wherein said decreasing expression or activity of endothelial genes and said increasing expression or activity of hematopoietic genes is conducted by increasing the expression or activity of Dnmt3b at an effective level and duration.

7. The method of claim 6, wherein increasing the expression or activity of Dnmt3b comprises one or more of introducing an encoding mRNA or an mRNA derivative, introducing an encoding transgene or episome, and introducing a genetic modification of expression elements or gain-of-function mutation to the Dnmt3b gene.

8. The method of any one of claims 1 to 3, wherein said decreasing expression or activity of endothelial genes and said increasing expression or activity of hematopoietic genes is conducted by increasing the expression or activity of Gimap6 at an effective level and duration.

9. The method of claim 8, wherein increasing the expression or activity of Gimap6 comprises one or more of introducing an encoding mRNA or an mRNA derivative, introducing an encoding transgene or an episome, and introducing a genetic modification of expression elements or gain-of-function mutation to the Gimap6 gene.

10. The method of any one of claims 1 to 3, wherein the said decreasing expression or activity of endothelial genes and said increasing expression or activity of hematopoietic genes is conducted by contacting the endothelial cells or HE cells with 2D or 3D cyclic strain, an agonist of Piezo1 at an effective concentration and duration, or combinations thereof.

11. The method of claim 10, wherein the Piezo1 agonist is Yoda1, Jedi1, and/or Jedi2.

12. The method of claim 11, wherein the effective amount of the Piezo1 agonist is in the range of 0.1 to 500 uM, or in the range of 0.1 to 100 μM.

13. The method of claim 10, wherein the agonist of Piezo1 is identified in a chemical library, based on: decreasing expression or activity of endothelial genes selected from vegfa, hey2, grp116, gnal3, sox17, cdh5, plxnd1, bcl6, and apln in endothelial and/or HE cells; and increasing expression or activity of two or more hematopoietic genes selected from runx1, spi1, cebpa, tal1, gfi1, gata2, and mllt3 in endothelial and/or HE cells upon contact with a candidate compound.

14. The method of any one of claims 1 to 13, wherein the method comprises providing a population comprising embryonic bodies, endothelial cells, hemogenic endothelial (HE) cells, or combinations thereof to a bioreactor.

15. The method of claim 14, wherein the bioreactor provides a cyclic-strain biomechanical stretching, an agonist of Piezo1 at an effective concentration and duration, or combinations thereof.

16. The method of claim 15, wherein the cyclic-strain biomechanical stretching decreases expression or activity of three or more endothelial genes selected from vegfa, hey2, grp116, gna13, sox17, cdh5, plxnd1, bcl6, and apln in the endothelial and/or HE cells; and increases expression or activity of two or more hematopoietic genes selected from runx1, spi1, cebpa, tal1, gfi1, gata2, and mllt3 in the endothelial and/or HE cells, so as to stimulate formation, and optionally expansion, of the HSCs.

17. The method of any one of claims 1 to 16, wherein the HSCs engraft in a hematopoietic niche and reconstitute to functional, multi-lineage adult blood.

18. The method of any one of claims 1 to 17, wherein HE cells are obtained from induced pluripotent stem cells (iPSCs), non-hematopoietic stem cells, somatic cells, or endothelial cells.

19. The method of any one of claims 1 to 18, wherein the hematopoietic stem cells comprise at least 1% long term hematopoietic stem cells (LT-HSCs), or at least 5% LT-HSCs.

20. The method of claim 19, wherein the hematopoietic stem cells comprise at least 0.1% long term hematopoietic stem cells (LT-HSCs).

21. The method of any one of claims 1 to 20, wherein the endothelial and/or HE cells are derived from HLA-modified or HLA-null cells, and/or transgene-free cells, gene-corrected, transgene-overexpressed, and are optionally derived by genetic or chemical induction of iPS cells or somatic cells.

22. The method of any one of claims 1 to 21, wherein source cells are obtained or derived from a subject, off-the-shelf library of cells, wherein the subject is optionally a universally compatible donor.

23. The method of claim 22, wherein the source cells are obtained or derived from a subject who has a blood, bone marrow, lysosomal storage, mitochondrial, metabolic, or immune disease.

24. The method of claim 23, wherein the subject does not have a hematological or non-hematological malignancy.

25. The method of any one of claims 1 to 24, further comprising, recovering and optionally expanding the HSCs.

26. The method of claim 25, wherein the population of HSCs are administered to a recipient, wherein the recipient is optionally the donor subject.

27. The method of claim 26, wherein at least about 10² HSCs are administered.

28. The method of claim 26, wherein at least about 10³ HSCs are administered.

29. The method of claim 26, wherein at least about 10⁴ HSCs are administered.

30. The method of claim 26, wherein at least about 10⁵ HSCs are administered.

31. A method for transitioning a population of cells to hematopoietic endothelial (HE) cells, the method comprising:
providing a population comprising embryonic bodies or endothelial cells, and providing a genetic, pharmacological, and/or mechanical stimulus selected from one or more of:
decreasing expression or activity of one or more endothelial genes selected from vegfa, hey2, grp116, gnal3, sox17, cdh5, plxnd1, bcl6, and apln in the cells; and increasing expression or activity of two or more hematopoietic genes selected from runx1, spi1, cebpa, tal1, gfi1, gata2 and mllt3 in the cells;
applying a 2D or 3D cyclic strain, and
contacting the cells with an agonist of Piezo1 at an effective concentration and duration; so as to transition the cells to HE cells.

32. The method of claim 31, wherein the increasing of activity or expression of the hematopoietic genes comprises one or more of: introducing an encoding mRNA or an mRNA derivative, introducing an encoding transgene or episome, introducing a genetic modification of expression elements; and introducing gain-of-function mutation to the hematopoietic gene.

33. The method of claim 32, wherein the decreasing of expression or activity of endothelial genes comprises one or more of: introducing a full or partial gene deletion, RNA silencing; anti sense oligonucleotide inhibition, pharmacological inhibition, introducing a genetic modification of expression elements, and introducing a loss-of-function mutation to the endothelial gene.

34. The method of claim 33 or 33, wherein said decreasing expression or activity of endothelial genes and said increasing expression or activity of hematopoietic genes is conducted by increasing the expression or activity of Dnmt3b at an effective level and duration.

35. The method of claim 34, wherein increasing the expression or activity of Dnmt3b comprises one or more of introducing an encoding mRNA or an mRNA derivative, introducing an encoding transgene or episome, introducing a genetic modification of expression elements, and incorporating a gain-of-function mutation to the Dnmt3b gene.

36. The method of any one of claims 32 to 35, wherein said decreasing expression or activity of endothelial genes and said increasing expression or activity of hematopoietic genes is conducted by increasing the expression or activity of Gimap6 at an effective level and duration.

37. The method of claim 36, wherein increasing the expression or activity of Gimap6 comprises one or more of: introducing an encoding mRNA or an mRNA derivative, introducing an encoding transgene or an episome, introducing a genetic modification of expression elements, and introducing gain-of-function mutation(s) to the Gimap6 gene.

38. The method of claim 31, wherein the cells are contacted with an effective amount of a Piezo1 agonist selected from Yoda1, Jedi1, and/or Jedi2.

39. The method of claim 38, wherein the effective amount of the Piezo1 agonist is in the range of 0.1 to 500 uM, or in the range of 0.1 to 100 μM.

40. The method of any one of claims 31 to 39, wherein the method comprises providing the population to a bioreactor, where the bioreactor provides a cyclic-strain biomechanical stretching.

41. The method of claim 40, wherein the HE cells are recovered, or are transitioned to HSCs, optionally by applying said genetic, pharmacological, and/or mechanical stimulus.

42. The method of claim 41, wherein the HE cells are transitioned to HSCs that engraft in a hematopoietic niche and reconstitute to functional, multi-lineage adult blood.

43. The method of any one of claims 31 to 42, wherein embryonic bodies or endothelial cells are derived from induced pluripotent stem cells (iPSCs).

44. The method of any one of claims 31 to 42, wherein the endothelial cells are derived from non-hematopoietic stem cells.

45. The method of any one of claims 31 to 44, wherein the HE cells are transitioned to hematopoietic stem cells comprising long term hematopoietic stem cells (LT-HSCs).

46. The method of any one of claims 31 to 45, wherein the population of cells are derived from HLA-modified or HLA-null cells.

47. The method of any one of claims 31 to 45, wherein the population of cells are transgene-free cells.

48. The method of any one of claims 31 to 47, further comprising expanding the HSCs in a process that comprises applying said genetic, pharmacological, and/or mechanical stimulus to the HSC cells.

49. The method of claim 48, wherein the HSCs are administered to a recipient, wherein the recipient is optionally the donor subject.

50. A method for expanding a population of hematopoietic stem cells (HSCs), the method comprising:
providing a population of HSCs, and providing a genetic, pharmacological, and/or mechanical stimulus selected from one or more of:
decreasing expression or activity of one or more endothelial genes selected from vegfa, hey2, grp116, gnal3, sox17, cdh5, plxnd1, bcl6, and apln in the cells; and increasing expression or activity of two or more hematopoietic genes selected from runx1, spi1, cebpa, tal1, gfi1, gata2 and mllt3 in the cells;
applying a 2D or 3D cyclic strain, and
contacting the cells with an agonist of Piezo1 at an effective concentration and duration; so as to expand the HSCs.

51. The method of claim 50, wherein the increasing of activity or expression of the hematopoietic genes comprises one or more of: introducing an encoding mRNA or an mRNA derivative, introducing an encoding transgene or episome, introducing a genetic modification of expression elements; and introducing gain-of-function mutation to the hematopoietic gene.

52. The method of claim 51, wherein the decreasing of expression or activity of endothelial genes comprises one or more of: introducing a full or partial gene deletion, RNA silencing, antisense oligonucleotide inhibition, pharmacological inhibition, introducing a genetic modification of expression elements, and introducing a loss-of-53 mutation to the endothelial gene.

53. The method of claim 51 or 52, wherein said decreasing expression or activity of endothelial genes and said increasing expression or activity of hematopoietic genes is conducted by increasing the expression or activity of Dnmt3b at an effective level and duration.

54. The method of claim 53, wherein increasing the expression or activity of Dnmt3b comprises one or more of introducing an encoding mRNA or an mRNA derivative, introducing an encoding transgene or episome, introducing a genetic modification of expression elements; and incorporating a gain-of-function mutation to the Dnmt3b gene.

55. The method of any one of claims 50 to 54, wherein said decreasing expression or activity of endothelial genes and said increasing expression or activity of hematopoietic genes is conducted by increasing the expression or activity of Gimap6 at an effective level and duration.

56. The method of claim 55, wherein increasing the expression or activity of Gimap6 comprises one or more of: introducing an encoding mRNA or an mRNA derivative, introducing an encoding transgene or an episome, introducing a genetic modification of expression elements, and introducing gain-of-function mutation(s) to the Gimap6 gene.

57. The method of claim 50, wherein the cells are contacted with an effective amount of a Piezo1 agonist selected from Yoda1, Jedi1, and/or Jedi2.

58. The method of claim 57, wherein the effective amount of the Piezo1 agonist is in the range of 0.1 to 500 uM, or in the range of 0.1 to 100 μM.

59. The method of any one of claims 50 to 58, wherein the method comprises providing the population to a bioreactor, where the bioreactor provides a cyclic-strain biomechanical stretching.

60. The method of claim 59, wherein the HSCs engraft in a hematopoietic niche and reconstitute to functional, multi-lineage adult blood.

61. The method of any one of claims 50 to 60, wherein the HSCs are transitioned from endothelial cells or HE cells.

62. The method of any one of claims 50 to 61, wherein the HSCs comprise long term hematopoietic stem cells (LT-HSCs).

63. The method of any one of claims 50 to 62, wherein the population of HSCs are derived from HLA-modified or HLA-null cells.

64. The method of claim 63, wherein the population of cells are transgene-free cells.

65. A pharmaceutical composition comprising a population of HSCs prepared by the method of any one of claims 1 to 64, and a pharmaceutically acceptable vehicle.

66. The pharmaceutical composition of claim 65, comprising at least 10⁴ LT-HSC cells.

67. A method of treating a subject in need of hematopoietic stem cell therapy or transplantation, the method comprising administering to the subject a therapeutically effective amount of hematopoietic stem cells (HSCs) prepared by the method of any one of claims 1 to 64 or administering the pharmaceutical composition of claim 65 or 66.

68. The method of claim 67, wherein the subject has malignant or non-malignant form of blood, bone marrow, lysosomal storage, mitochondrial, metabolic, or immune disease.

69. The method of claim 67 or 68, wherein the subject has a condition selected from acute lymphoblastic leukemia, chronic myeloid leukemia, chronic lymphocytic leukemia, a myeloproliferative disorder; myelodysplastic syndrome; multiple myeloma; non-Hodgkin lymphoma, Hodgkin disease, neuroblastoma, a germ cell tumor, or amyloidosis.

70. The method of claim 69, wherein the subject has a condition selected from an autoimmune disorder such as systemic lupus erythematosus (SLE) or systemic sclerosis; aplastic anemia; pure red-cell aplasia; paroxysmal nocturnal hemoglobinuria, Fanconi anemia; thalassemia major; sickle cell anemia; severe combined immunodeficiency (SCID); Wiskott-Aldrich syndrome; Hemophagocytic lymphohistiocytosis; inborn errors of metabolism; epidermolysis bullosa; severe congenital neutropenia; Shwachman-Diamond syndrome; Diamond-Blackfan anemia; Pearson Syndrome, and Leukocyte adhesion deficiency.

71. A method for making hematopoietic stem cells (HSCs), comprising:
contacting a panel of chemical compounds with embryonic bodies, endothelial cells and/or hemogenic endothelial cells, and determining a change in expression level induced by said chemical compounds of: Dnmt3b or Gimap6; at least two of vegfa, hey2, grp116, gnal3, sox17, cdh5, plxnd1, bcl6, and apln; and at least two of runx1, spi1, cebpa, tal1, gfi1, gata2, and mllt3;
selecting a compound that induces the following changes in gene expression:
increase in expression of Dnmt3b and/or Gimap6,
decrease in expression of two of more of vegfa, hey2, grp116, gna13, sox17, cdh5, plxnd1, bcl6, and apln; and
increase in expression of two or more runx1, spi1, cebpa, tal1, gfi1, gata2, and mllt3; and
inducing the transition of endothelial cells and/or hemogenic endothelial cells to HSCs by contacting the selected compound with endothelial cells and/or hemogenic endothelial cells, thereby making self-renewing HSCs that can engraft and reconstitute multi-lineage adult blood.

72. The method of claim 71, wherein the selected compound decreases expression of five or more endothelial genes selected from vegfa, hey2, grp116, gnal3, sox17, cdh5, plxnd1, bcl6, and apln in the endothelial and/or HE cells; and increases expression of five or more hematopoietic genes selected from runx1, spi1, cebpa, tal1, gfi1, gata2, and mllt3 in the endothelial and/or HE cells, so as to stimulate formation and optionally expansion of the HE cells, HSCs, or combinations thereof.

73. The method of claim 72, wherein the selected compound decreases expression of vegfa, hey2, grp116, gna13, cdh5, and plxnd1 in the endothelial and/or HE cells; and increases expression of runx1, spi1, cebpa, tal1, and gata2 in the endothelial and/or HE cells.

74. The method of any one of claims 71 to 73, wherein the selected compound increases the expression of Dnmt3b.

75. The method of any one of claims 71 to 73, wherein the selected compound is a Piezo1 agonist.

76. The method of claim 74, wherein the selected compound is a derivative of Yoda1, Jedi1, and/or Jedi2.

77. The method of any one of claims 71 to 76, wherein embryonic bodies, endothelial cells or HE cells are obtained from induced pluripotent stem cells (iPSCs), non-hematopoietic stem cells, somatic cells, or endothelial cells.

78. The method of any one of claims 71 to 76, wherein the hematopoietic stem cells obtained comprise at least 0.1% long term hematopoietic stem cells (LT-HSCs).

79. The method of claim 78, wherein the hematopoietic stem cells obtained comprise at least about 1% or at least about 10% long term hematopoietic stem cells (LT-HSCs).

80. The method of claim 78, wherein the hematopoietic stem cells obtained comprise from about 2% to about 25% LT-HSCs.

81. The method of any one of claims 71 to 80, wherein the endothelial and/or HE cells are derived from HLA-modified or HLA-null cells, transgene-overexpressed, and/or transgene-free cells, and are optionally derived by genetic or chemical induction of iPS cells or somatic cells.

82. The method of any one of claims 71 to 81, wherein source cells are obtained or derived from a subject, wherein the subject is optionally a patient, matched or unmatched donor, or a universally compatible donor.

83. The method of any one of claims 71 to 82, further comprising, recovering the HSCs.

84. A composition for cellular therapy produced according to this disclosure, and comprising at least about 10³ or at least about 10⁴ LT-HSC cells.
